SEQUENCE LISTING (7 sequences)

SEQ ID NO: 1 - protein, 126 aa, Lama glama
QLQLVESGGGLVQPGGSLRLSCAASTLTLNSYAIGWFRQAPGKEREAVSCTSSSGDMTYYANSVKGRFTISRDNAKNTVYLQMSRLKPEDTAVYYCAAVKLEYGYVCSHPNEYDYWGQGTQVTVSS

SEQ ID NO: 2 - protein, 126 aa, Lama glama
QLQLVESGGGLVQPGGSLRLSCAASTLTLNSYAIGWFRQAPGKEREAVSCTSSSGDMTYYTNSVKGRFTISRDNAKNTVYLQMSRLKPDDTAVYYCAAVLLEYGYVCSHPNEYDYWGQGTQVTVSS

SEQ ID NO: 3 - protein, 5 aa, Lama glama
SYAIG

SEQ ID NO: 4 - protein, 17 aa, Lama glama
CTSSSGDMTYYANSVKG

SEQ ID NO: 5 - protein, 17 aa, Lama glama
VKLEYGYVCSHPNEYDY

SEQ ID NO: 6 - protein, 16 aa, Lama glama
CTSSSGDMTYYTNSVK

SEQ ID NO: 7 - protein, 17 aa, Lama glama
VLLEYGYVCSHPNEYDY

(19) United States
(12) Patent Application Publication
(10) Pub. No.: US 2024/0190947 A1
(43) Pub. Date: Jun. 13, 2024

(54) CAMELID ANTI-SEVERE ACUTE RESPIRATORY SYNDROME CORONAVIRUS ANTIBODIES

(71) Applicants: Ernesto Rubin RESNIK, Minneapolis, MN (US); Anna Agnieszka BAJER, Minneapolis, MN (US); Cyrus B MUNSHI, Minneapolis, MN (US); Kerri Ann BOSTROM, Minneapolis, MN (US); Jody BONNEVIER, Minneapolis, MN (US); BIO-TECHNE CORPORATION, Minneapolis, MN (US)

(72) Inventors: Ernesto R. Resnik, Minneapolis, MN (US); Anna Agnieszka Bajer, New Brighton, MN (US); Cyrus B. Munshi, Blaine, MN (US); Kerri Ann Bostrom, Stanchfield, MN (US); Jody Bonnevier, Chanhassen, MN (US); Christine Andrea Goetz, Blaine, MN (US)

(21) Appl. No.: 18/286,852

(22) PCT Filed: Apr. 7, 2022
(86) PCT No.: PCT/US2022/023783

§ 371 Date: Oct. 13, 2023

Related U.S. Application Data
(60) Provisional application No. 63/175,636, filed on Apr. 16, 2021.

(51) Int. Cl. C07K 16/10; A61K 49/00; A61P 31/14

(52) U.S. Cl. CPC C07K 16/1003; A61K 49/0058; A61P 31/14; C07K 2317/22; C07K 2317/31; C07K 2317/35; C07K 2317/569; C07K 2317/76

(57) ABSTRACT

Anti-SARS-CoV-specific llama single domain antibodies, methods of making and characterizing those antibodies, and methods of using those antibodies are described herein. In some embodiments, the antibodies may bind to both SARS-CoV-1 and SARS-CoV-2. In some embodiments, the antibodies bind to S₁ of the spike protein of SARS-CoV-2 including, for example, to the receptor binding domain (RBD) of S₁. In some embodiments, the antibodies may block the binding of SARS-CoV-1 and/or SARS-CoV-2 to ACE-2.

### CONTINUING APPLICATION DATA

This application claims the benefit of U.S. Provisional Application Ser. No. 63/175,636, filed Apr. 16, 2021, which is incorporated by reference herein.

### SEQUENCE LISTING

This application contains a Sequence Listing electronically submitted via EFS-Web to the United States Patent and Trademark Office as an ASCII text file entitled “0541-000016WO01_ST25.txt” having a size of 4 kilobytes and created on Mar. 12, 2022. The information contained in the Sequence Listing is incorporated by reference herein.

### BACKGROUND

Severe acute respiratory syndrome coronaviruses (SARS-CoV) including SARS-CoV-1 and SARS-CoV-2 are RNA viruses that are members of the coronaviridae family. SARS-CoV-1 and SARS-CoV-2 are the etiological agents of severe acute respiratory syndrome (SARS) and COVID-19, respectively. Both SARS-CoV-1 and SARS-CoV-2 primarily infect vascular endothelial cells of the human respiratory system by binding to the angiotensin converting enzyme 2 (ACE-2) via the virus's heterodimeric spike protein. The SARS-coronavirus spike protein is composed of two subunits: S₁ and S₂, with the S₁ being primarily responsible for binding to the host cell receptor (ACE-2) via a highly conserved receptor binding domain (RBD).

Highly characterized antibodies that are capable of binding to and blocking the binding of SARS-coronavirus spike protein to human ACE-2 may be useful for elucidating SARS-coronavirus/host interactions, for identifying target epitopes on the SARS-coronavirus spike protein, for prevention of a SARS-coronavirus infection, for development of assays to detect the presence of SARS-coronavirus in clinical specimens, and for therapeutic treatment of COVID-19.

### SUMMARY OF THE INVENTION

The present invention includes an antigen binding molecule with an antibody variable domain having an amino acid sequence with at least 75%, at least 80%, at least 85%, at least 90%, at least 95%, at least 96%, at least 97%, at least 98%, or at least 99% sequence identity to QLQLVESGGGLVQPGGSLRLSCAASTLTLNSYAIGWFRQAPGKEREAVSCTSSSGDMTYY ANSVKGRFTISRDNAKNTVYLQMSRLKPEDTAVYYCAAVKLEYGYVCSSHPNEYDYWGQG TQVTVSS (SEQ ID NO: 1); or with at least 75%, at least 80%, at least 85%, at least 90%, at least 95%, at least 96%, at least 97%, at least 98%, or at least 99% sequence identity to

(SEQ ID NO: 2)
QLQLVESGGGLVQPGGSLRLSCAASTLTLNSYAIGWFRQAPGKEREAVS
CTSSSGDMTYYTNSVKGRFTISRDNAKNTVYLQMSRLKPDDTAVYYCAA
VLLEYGYVCSHPNEYDYWGQGTQVTVSS.

In some aspects, the antigen binding molecule includes a variable domain with an amino acid sequence including

(SEQ ID NO: 1)
QLQLVESGGGLVQPGGSLRLSCAASTLTLNSYAIGWFRQAPGKEREAVS
CTSSSGDMTYYANSVKGRFTISRDNAKNTVYLQMSRLKPEDTAVYYCAA
VKLEYGYVCSHPNEYDYWGQGTQVTVSS;
or
(SEQ ID NO: 2)
QLQLVESGGGLVQPGGSLRLSCAASTLTLNSYAIGWFRQAPGKEREAVS
CTSSSGDMTYYTNSVKGRFTISRDNAKNTVYLQMSRLKPDDTAVYYCAA
VLLEYGYVCSHPNEYDYWGQGTQVTVSS.

The present invention includes an antigen binding molecule having an antibody variable domain having one or more complementary determining regions (CDRs) selected from:
a CDR1 sequence having an amino acid sequence which at least 75% sequence identity to SYAIG (SEQ ID NO: 3);
a CDR 2 sequence having an amino acid sequence with at least 75%, at least 80%, at least 85%, at least 90%, at least 95%, at least 96%, at least 97%, at least 98%, or at least 99% sequence identity to CTSSSGDMTYYANSVKG (SEQ ID NO: 4) or at least 75%, at least 80%, at least 85%, at least 90%, at least 95%, at least 96%, at least 97%, at least 98%, or at least 99% sequence identity to CTSSSGDMTYYTNSVKG (SEQ ID NO: 6); and/or
a CDR3 sequence having an amino acid sequence with at least 75%, at least 80%, at least 85%, at least 90%, at least 95%, at least 96%, at least 97%, at least 98%, or at least 99% sequence identity to VKLEYGYVCSHIPNEYDY (SEQ ID NO: 5) or at least 75%, at least 80%, at least 85%, at least 90%, at least 95%, at least 96%, at least 97%, at least 98%, or at least 99% sequence identity to VLLEYGYVCSHPNEYDY (SEQ ID NO: 7).

The present invention includes an antigen binding molecule having an antibody variable domain having one or more complementary determining regions (CDRs) selected from:
a CDR1 sequence having an amino acid sequence which is identical to, or differs by one, two, or three amino acids from, SYAIG (SEQ ID NO: 3);
a CDR2 sequence having an amino acid sequence which is identical to, or differs by one, two, three, four, or five amino acids from, CTSSSGDMTYYANSVKG (SEQ ID NO: 4) or which is identical to, or differs by one, two, three, four, or five amino acids from CTSSSGDMTYYTNSVKG (SEQ ID NO: 6); and/or
a CDR3 sequence having an amino acid sequence which is identical to, or differs by one, two, three, four, or five amino acids from, VKLEYGYVCSHPNEYDY (SEQ ID NO: 5) or which is identical to, or differs by one, two, three, four, or five amino acids from

(SEQ ID NO: 7)
VLLEYGYVCSHPNEYDY.

In some aspects, the antigen binding molecule includes an antibody variable domain having: a CDR1 sequence having the amino acid sequence SYAIG (SEQ ID NO: 3); a CDR2 sequence having the amino acid sequence CTSSSGDMTYYANSVKG (SEQ ID NO: 4) or CTSSSGDMTYYTNSVKG (SEQ ID NO: 6); and a CDR3 sequence having the amino acid sequence VKLEYGYVCSHPNEYDY (SEQ ID NO: 5) or VLLEYGYVCSHPNEYDY (SEQ ID NO: 7).

In some aspects, the antigen binding molecule includes an antibody variable domain having: a CDR1 sequence having the amino acid sequence SYAIG (SEQ ID NO: 3); a CDR2 sequence having the amino acid sequence CTSSSGDMTYYANSVKG (SEQ ID NO: 4); and a CDR3 sequence having the amino acid sequence VKLEYGYVCSHPNEYDY (SEQ ID NO: 5); or having a CDR1 sequence having the amino acid sequence SYAIG (SEQ ID NO: 3); a CDR2 sequence having the amino acid sequence CTSSSGDMTYYTNSVKG (SEQ ID NO: 6); and a CDR3 sequence having the amino acid sequence VLLEYGYVCSHPNEYDY (SEQ ID NO: 7).

The present invention includes an antibody variable domain having an amino acid sequence with at least 75%, at least 80%, at least 85%, at least 90%, at least 95%, at least 96%, at least 97%, at least 98%, or at least 99% sequence identity to

(SEQ ID NO: 1)
QLQLVESGGGLVQPGGSLRLSCAASTLTLNSYAIGWFRQAPGKEREAVS
CTSSSGDMTYYANSVKGRFTISRDNAKNTVYLQMSRLKPEDTAVYYCAA
VKLEYGYVCSHPNEYDYWGQGTQVTVSS
or
(SEQ ID NO: 2)
QLQLVESGGGLVQPGGSLRLSCAASTLTLNSYAIGWFRQAPGKEREAVS
CTSSSGDMTYYTNSVKGRFTISRDNAKNTVYLQMSRLKPDDTAVYYCAA
VLLEYGYVCSHPNEYDYWGQGTQVTVSS.

In some aspects, the antibody variable domain includes an amino acid sequence having

(SEQ ID NO: 1)
QLQLVESGGGLVQPGGSLRLSCAASTLTLNSYAIGWFRQAPGKEREAVS
CTSSSGDMTYYANSVKGRFTISRDNAKNTVYLQMSRLKPEDTAVYYCAA
VKLEYGYVCSHPNEYDYWGQGTQVTVSS
or
(SEQ ID NO: 2)
QLQLVESGGGLVQPGGSLRLSCAASTLTLNSYAIGWFRQAPGKEREAVS
CTSSSGDMTYYTNSVKGRFTISRDNAKNTVYLQMSRLKPDDTAVYYCAA

The present invention includes an antibody variable domain having one or more complementary determining regions (CDRs) selected from:
a CDR1 sequence having an amino acid sequence which at least 75% sequence identity to SYAIG (SEQ ID NO: 3);
a CDR 2 sequence having an amino acid sequence with at least 75%, at least 80%, at least 85%, at least 90%, at least 95%, at least 96%, at least 97%, at least 98%, or at least 99% sequence identity to CTSSSGDMTYYANSVKG (SEQ ID NO: 4) or at least 75%, at least 80%, at least 85%, at least 90%, at least 95%, at least 96%, at least 97%, at least 98%, or at least 99% sequence identity to CTSSSGDMTYYTNSVKG (SEQ ID NO: 6); and
a CDR3 sequence having an amino acid sequence with at least 75%, at least 80%, at least 85%, at least 90%, at least 95%, at least 96%, at least 97%, at least 98%, or at least 99% sequence identity to VKLEYGYVCSHPNEYDY (SEQ ID NO: 5) or at least 75%, at least 80%, at least 85%, at least 90%, at least 95%, at least 96%, at least 97%, at least 98%, or at least 99% sequence identity to VLLEYGYVCSHPNEYDY (SEQ ID NO: 7).

The present invention includes an antibody variable domain having one or more complementary determining regions (CDRs) selected from:
a CDR1 sequence having an amino acid sequence which is identical to, or differs by one, two, or three amino acids from, SYAIG (SEQ ID NO: 3);
a CDR2 sequence having an amino acid sequence which is identical to, or differs by one, two, three, four, or five amino acids from, CTSSSGDMTYYANSVKG (SEQ ID NO: 4) or which is identical to, or differs by one, two, three, four, or five amino acids from CTSSSGDMTYYTNSVKG (SEQ ID NO: 6); and
a CDR3 sequence having an amino acid sequence which is identical to, or differs by one, two, three, four, or five amino acids from, VKLEYGYVCSHPNEYDY (SEQ ID NO: 5) or which is identical to, or differs by one, two, three, four, or five amino acids from

(SEQ ID NO: 7)
VLLEYGYVCSHPNEYDY.

In some aspects, the antibody variable domain includes: a CDR1 sequence having the amino acid sequence SYAIG (SEQ ID NO: 3); a CDR2 sequence having the amino acid sequence CTSSSGDMTYYANSVKG (SEQ ID NO: 4) or CTSSSGDMTYYTNSVKG (SEQ ID NO: 6); and
a CDR3 sequence having the amino acid sequence VKLEYGYVCSHPNEYDY (SEQ ID NO: 5) or VLLEYGYVCSHPNEYDY (SEQ ID NO: 7).

In some aspects, the antibody variable domain having:
a CDR1 sequence having the amino acid sequence SYAIG (SEQ ID NO: 3); a CDR2 sequence having the amino acid sequence CTSSSGDMTYYANSVKG (SEQ ID NO: 4); and a CDR3 sequence having the amino acid sequence VKLEYGYVCSHPNEYDY (SEQ ID NO: 5); or having:
a CDR1 sequence having the amino acid sequence SYAIG (SEQ ID NO: 3); a CDR2 sequence having the amino acid sequence CTSSSGDMTYYTNSVKG (SEQ ID NO: 6); and a CDR3 sequence comprising the amino acid sequence VLLEYGYVCSHPNEYDY (SEQ ID NO: 7).

The present invention includes a camelid antibody single variable domain having an amino acid sequence with at least 75%, at least 80%, at least 85%, at least 90%, at least 95%, at least 96%, at least 97%, at least 98%, or at least 99% sequence identity to

(SEQ ID NO: 1)
QLQLVESGGGLVQPGGSLRLSCAASTLTLNSYAIGWFRQAPGKEREAVS
CTSSSGDMTYYANSVKGRFTISRDNAKNTVYLQMSRLKPEDTAVYYCAA
VKLEYGYVCSHPNEYDYWGQGTQVTVSS
or
(SEQ ID NO: 2)
QLQLVESGGGLVQPGGSLRLSCAASTLTLNSYAIGWFRQAPGKEREAVS
CTSSSGDMTYYTNSVKGRFTISRDNAKNTVYLQMSRLKPDDTAVYYCAA
VLLEYGYVCSHPNEYDYWGQGTQVTVSS.

In some aspects, the camelid antibody single variable domain includes an amino acid sequence having

(SEQ ID NO: 1)
QLQLVESGGGLVQPGGSLRLSCAASTLTLNSYAIGWFRQAPGKEREAVS
CTSSSGDMTYYANSVKGRFTISRDNAKNTVYLQMSRLKPEDTAVYYCAA
VKLEYGYVCSHPNEYDYWGQGTQVTVSS
or
(SEQ ID NO: 2)
QLQLVESGGGLVQPGGSLRLSCAASTLTLNSYAIGWFRQAPGKEREAVS
CTSSSGDMTYYTNSVKGRFTISRDNAKNTVYLQMSRLKPDDTAVYYCAA
VLLEYGYVCSHPNEYDYWGQGTQVTVSS.

The present invention includes a camelid antibody single variable domain having one or more complementary determining regions (CDRs) selected from: a CDR1 sequence having an amino acid sequence which at least 75% sequence identity to SYAIG (SEQ ID NO: 3); a CDR 2 sequence having an amino acid sequence with at least 75%, at least 80%, at least 85%, at least 90%, at least 95%, at least 96%, at least 97%, at least 98%, or at least 99% sequence identity to CTSSSGDMTYYANSVKG (SEQ ID NO: 4) or at least 75%, at least 80%, at least 85%, at least 90%, at least 95%, at least 96%, at least 97%, at least 98%, or at least 99% sequence identity to CTSSSGDMTYYTNSVKG (SEQ ID NO: 6); and a CDR3 sequence having an amino acid sequence with at least 75%, at least 80%, at least 85%, at least 90%, at least 95%, at least 96%, at least 97%, at least 98%, or at least 99% sequence identity to VKLEYGYVCSHIPNEYDY (SEQ ID NO: 5) or at least 75%, at least 80%, at least 85%, at least 90%, at least 95%, at least 96%, at least 97%, at least 98%, or at least 99% sequence identity to VLLEYGYVCSHPNEYDY (SEQ ID NO: 7).

The present invention includes a camelid antibody single variable domain having one or more complementary determining regions (CDRs) selected from:
a CDR1 sequence having an amino acid sequence which is identical to, or differs by one, two, or three amino acids from, SYAIG (SEQ ID NO: 3);
a CDR2 sequence having an amino acid sequence which is identical to, or differs by one, two, three, four, or five amino acids from, CTSSSGDMTYYANSVKG (SEQ ID NO: 4) or which is identical to, or differs by one, two, three, four, or five amino acids from CTSSSGDMTYYTNSVKG (SEQ ID NO: 6); and/or
a CDR3 sequence having an amino acid sequence which is identical to, or differs by one, two, three, four, or five amino acids from, VKLEYGYVCSHPNEYDY (SEQ ID NO: 5) or which is identical to, or differs by one, two, three, four, or five amino acids from

(SEQ ID NO: 7)
VLLEYGYVCSHPNEYDY.

In some aspects, the camelid antibody single variable domain includes a CDR1 sequence having the amino acid sequence SYAIG (SEQ ID NO: 3); a CDR2 sequence having the amino acid sequence CTSSSGDMTYYANSVKG (SEQ ID NO: 4) or CTSSSGDMTYYTNSVKG (SEQ ID NO: 6); and a CDR3 sequence having the amino acid sequence VKLEYGYVCSHPNEYDY (SEQ ID NO: 5) or VLLEYGYVCSHPNEYDY (SEQ ID NO: 7).

In some aspects, the camelid antibody single variable domain includes:
a complementary determining region 1 (CDR1) sequence having the amino acid sequence SYAIG (SEQ ID NO: 3); a CDR2 sequence having the amino acid sequence CTSSSGDMTYYANSVKG (SEQ ID NO: 4); and a CDR3 sequence having the amino acid sequence VKLEYGYVCSHPNEYDY (SEQ ID NO: 5); or
a CDR1 sequence having the amino acid sequence SYAIG (SEQ ID NO: 3); a CDR2 sequence having the amino acid sequence CTSSSGDMTYYTNSVKG (SEQ ID NO: 6); and a CDR3 sequence having the amino acid sequence VLLEYGYVCSHPNEYDY (SEQ ID NO: 7).

The present invention includes a camelid heavy chain IgG (hcIgG) antibody having a camelid antibody single variable domain as described herein. In some aspects, the camelid heavy chain hcIgG is a llama heavy chain IgG (hcIgG) antibody.

In some aspects, an antibody binding molecule, antibody variable domain, camelid antibody single variable domain, or hcIgG antibody as described herein specifically binds the SARS-CoV-2 spike protein S1.

In some aspects, an antibody binding molecule, antibody variable domain, camelid antibody single variable domain, or hcIgG antibody as described herein specifically binds to the receptor binding (RBD) domain (amino acids 319 to 541) of the SARS-CoV-2 spike protein S1.

In some aspects, an antibody binding molecule, antibody variable domain, camelid antibody single variable domain, or hcIgG antibody as described herein inhibits binding of SARS-CoV-1 and/or SARS-CoV-2 to ACE-2. In some aspects, the binding of SARS-CoV-1 or SARS-CoV-2 or both SARS-CoV-1 and SARS-CoV-2 to ACE-2 is decreased by at least 10 percent (%), at least 15%, at least 20%, at least 25%, at least 30%, at least 40%, at least 50%, at least 60%, at least 70%, at least 80%, at least 90%, at least 95%, or at least 98%.

In some aspects, an antibody binding molecule, antibody variable domain, camelid antibody single variable domain, or hcIgG antibody as described herein is humanized.

In some aspects, an antibody binding molecule, antibody variable domain, camelid antibody single variable domain, or hcIgG antibody as described herein further includes a human immunoglobulin constant region.

In some aspects, an antibody binding molecule, antibody variable domain, camelid antibody single variable domain, or hcIgG antibody as described herein is conjugated to a small molecule or protein.

In some aspects, an antibody binding molecule, antibody variable domain, camelid antibody single variable domain, or hcIgG antibody as described herein is conjugated to a detectable marker.

The present invention includes a bispecific or multivalent antibody including an antibody binding molecule, antibody variable domain, camelid antibody single variable domain, or hcIgG antibody as described herein.

The present invention includes a composition including an antibody binding molecule, antibody variable domain, camelid antibody single variable domain, or hcIgG antibody as described herein. In some aspects, the composition includes at least one additional anti-SARS-CoV antibody. In some aspects, the composition includes a pharmaceutically acceptable carrier.

The present invention includes a method of administering the antibody binding molecule, antibody variable domain, camelid antibody single variable domain, or hcIgG antibody as described herein or a composition thereof to a subject. In some aspects, the subject is suspected of having SARS-CoV-1 or SARS-CoV-2 or has been diagnosed with SARS-CoV-1 or SARS-CoV-2. In some aspects, the subject has been exposed to SARS-CoV-1 or SARS-CoV-2. In some aspects, the subject is a human. In some aspects, the method includes administering multiple doses of the antibody binding molecule, antibody variable domain, camelid antibody single variable domain, or hcIgG antibody to a subject.

The present invention includes a method of using an antibody binding molecule, antibody variable domain, camelid antibody single variable domain antibody, or hcIgG antibody as described herein or a composition thereof to diagnose a subject with SARS-CoV-1 or SARS-CoV-2.

The present invention includes a nucleotide sequence encoding an antibody binding molecule, antibody variable domain, camelid antibody single variable domain, or hcIgG antibody as described herein. The present invention includes an expression vector having such a nucleotide sequence. The present invention includes a host cell transformed with such a nucleotide sequence or expression vector.

The present invention includes a method for producing an antibody binding molecule, antibody variable domain, camelid antibody single variable domain antibody, or hcIgG antibody, the method including culturing such a host cell under conditions that allow the host cell to translate the nucleotide sequence encoding the antibody binding molecule, camelid antibody single variable domain, or hcIgG antibody, thereby producing an antibody binding molecule, antibody variable domain, camelid antibody single variable domain antibody, or hcIgG antibody. In some aspects, the method further includes harvesting, purifying and/or isolating said antibody binding molecule, camelid antibody single variable domain antibody, or hcIgG antibody.

The term “antibody” as used herein refers to a molecule that contains at least one antigen binding site that immunospecifically binds to a particular antigen target of interest. The term “antibody” thus includes but is not limited to a full-length antibody and/or its variants, a fragment thereof, peptibodies and variants thereof, monoclonal antibodies (including full-length monoclonal antibodies), polyclonal antibodies, multispecific antibodies (for example, bispecific antibodies) formed from at least two intact antibodies, human antibodies, humanized antibodies, and antibody mimetics that mimic the structure and/or function of an antibody or a specified fragment or portion thereof, including single chain antibodies and fragments thereof. Binding of an antibody to a target can cause a variety of effects, such as but not limited to where such binding modulates, decreases, increases, antagonizes, agonizes, mitigates, alleviates, blocks, inhibits, abrogates and/or interferes with at least one target activity or binding, or with receptor activity or binding, in vitro, in situ, and/or in vivo. An antibody of the present disclosure thus encompasses antibody fragments capable of binding to a biological molecule (such as an antigen or receptor) or portions thereof, including but not limited to Fab, Fab′ and F(ab′)₂, pFc′, Fd, a single domain antibody (sdAb), a variable fragment (Fv), a single-chain variable fragment (scFv) or a disulfide-linked Fv (sdFv); a diabody or a bivalent diabody; a linear antibody; a single-chain antibody molecule; and a multispecific antibody formed from antibody fragments. The antibody may be of any type, any class, or any subclass. When the antibody is a human or mouse antibody, the type may include, for example, IgG, IgE, IgM, IgD, IgA and IgY, and the class may include, for example, IgG1, IgG2, IgG3, IgG4, IgA1 and IgA2. When the antibody is a camelid antibody, the type may include, for example, camelid single-chain antibodies (scAbs), camelid VHH region, and single domain antibody (VHH).

The term “monoclonal antibody” as used herein refers to an antibody obtained from a population of substantially homogeneous antibodies, that is, the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which typically include different antibodies directed against different determinants (epitopes), each monoclonal antibody is directed against a single determinant on the antigen. The monoclonal antibodies may be synthesized by hybridoma cells uncontaminated by other immunoglobulin producing cells. Alternatively, the monoclonal antibody may be produced recombinantly including, for example, by cells stably or transiently transfected with the heavy and light chain genes encoding the monoclonal antibody.

The modifier “monoclonal” indicates the character of an antibody, as defined above, as being obtained from a substantially homogeneous population of antibodies and is not to be construed as requiring engineering of the antibody by any particular method. In some embodiments, the term “monoclonal” is used herein to refers to an antibody that is derived from a clonal population of cells, including any eukaryotic, prokaryotic, or phage clone, and not the method by which the antibody was engineered.

As used herein, “isolated” refers to material removed from its original environment (for example, the natural environment if it is naturally occurring), and thus is altered “by the hand of man” from its natural state.

As used herein, “room temperature” is 16° C. to 26° C. or, more preferably, 18° C. to 24° C. In some embodiments, “room temperature” is 20° C. to 22° C.

As used herein “sequence identity” between two polypeptides is determined by comparing the amino acid sequence of one polypeptide to the sequence of a second polypeptide. When discussed herein, whether any particular polypeptide is at least 40 percent (%), at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% identical to another polypeptide may be determined using methods and computer programs/software known in the art such as, but not limited to, the BESTFIT program (Wisconsin Sequence Analysis Package, Version 8 for Unix, Genetics Computer Group, University Research Park, 575 Science Drive, Madison, Wis. 53711). BESTFIT uses the local homology algorithm of Smith and Waterman (1981) Advances in Applied Mathematics 2:482-489, to find the best segment of homology between two sequences. When using BESTFIT or any other sequence alignment program to determine whether a particular sequence is, for example, 95% identical to a reference sequence according to the present disclosure, the parameters are set such that the percentage of identity is calculated over the full-length of the reference polypeptide sequence and that gaps in homology of up to 5% of the total number of amino acids in the reference sequence are allowed.

“Binding affinity” or “affinity binding” refers to the strength of the sum total of noncovalent interactions between a single binding site of a molecule (for example, an antibody) and its binding partner (for example, an antigen or antigenic epitope). The affinity of a molecule X for its partner Y is represented by the dissociation constant (K_{D}), which can generally be determined by using methods known in the art, for example, using the BIACORE biosensor, commercially available from BIACORE (GE Healthcare Worldwide, Chicago, IL). In some embodiments, antibodies of the present disclosure may be described in terms of their binding affinity for SARS-CoV. In some embodiments, antibodies of the present disclosure include antibodies that interact with an antigen wherein the dissociation constant (K_{D}) is less than or equal to 5×10⁻⁶ M, less than or equal to 1×10⁻⁶ M, less than or equal to 5×10⁻⁷ M, less than or equal to 1×10⁻⁷ M, less than or equal to 5×10⁻⁸ M, less than or equal to 1×10⁻⁸ M, less than or equal to 5×10⁻⁹ M, less than or equal to 1×10⁻⁹ M, less than or equal to 5×10⁻¹⁰ M, less than or equal to 1×10⁻¹⁰ M, less than or equal to 5×10⁻¹¹ M, less than or equal to 1×10⁻¹¹ M, less than or equal to 5×10⁻¹² M, less than or equal to 1×10⁻¹² M, less than or equal to 5×10⁻¹³ M, less than or equal to 1×10⁻¹³ M, less than or equal to 5×10⁻¹⁴ M, less than or equal to 1×10⁻¹⁴ M, less than or equal to 5×10⁻¹⁵ M, or less than or equal to 1×10⁻⁵ M.

As used herein, the term “subject” includes, but is not limited to, humans and non-human vertebrates. In some embodiments, a subject is a mammal, particularly a human. A subject may be an individual. A subject may be an “individual,” “patient,” or “host.” Non-human vertebrates include livestock animals, companion animals, and laboratory animals. Non-human subjects also include non-human primates as well as rodents, such as, but not limited to, a rat or a mouse. Non-human subjects also include, without limitation, chickens, horses, cows, pigs, goats, dogs, cats, guinea pigs, hamsters, mink, and rabbits.

As used herein “in vitro” is in cell culture and “in vivo” is within the body of a subject.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

The terms “comprises” and variations thereof do not have a limiting meaning where these terms appear in the description and claims. Such terms will be understood to imply the inclusion of a stated step or element or group of steps or elements but not the exclusion of any other step or element or group of steps or elements.

By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

Unless otherwise specified, “a,” “an,” “the,” and “at least one” are used interchangeably and mean one or more than one.

As used herein, the term “or” is generally employed in its usual sense including “and/or” unless the content clearly dictates otherwise.

The term “and/or” means one or all of the listed elements or a combination of any two or more of the listed elements.

Also herein, the recitations of numerical ranges by endpoints include all numbers subsumed within that range (e.g., 1 to 5 includes 1, 1.5, 2, 2.75, 3, 3.80, 4, 5, etc.).

Herein, “up to” a number (for example, up to 50) includes the number (for example, 50).

The term “in the range” or “within a range” (and similar statements) includes the endpoints of the stated range.

For any method disclosed herein that includes discrete steps, the steps may be conducted in any feasible order. And, as appropriate, any combination of two or more steps may be conducted simultaneously.

All headings are for the convenience of the reader and should not be used to limit the meaning of the text that follows the heading, unless so specified.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure.

Furthermore, the particular features, configurations, compositions, or characteristics may be combined in any suitable manner in one or more embodiments.

Unless otherwise indicated, all numbers expressing quantities of components, molecular weights, and so forth used in the specification and claims are to be understood as being modified in all instances by the term “about.” As used herein in connection with a measured quantity, the term “about” refers to that variation in the measured quantity as would be expected by the skilled artisan making the measurement and exercising a level of care commensurate with the objective of the measurement and the precision of the measuring equipment used. Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Structure of human immunoglobulin, llama immunoglobulin, and a VHH single domain antibody.

FIGS. 2A-2E. A schematic of SARS-CoV-2 viral antibody blocking cell model, using a hACE-2 HEK/eGFP Tfx and rSARS-CoV-2 S₁ RBD protein for illustrative purposes. FIG. 2A shows a schematic of SARS-CoV-2 viral antibody blocking cell model. An antibody and a recombinant SARS-CoV-2 S₁ RBD-His protein are exposed to one another, allowing a complex to form between the antibody (for example, an anti-SARS-CoV-2 antibody) and the SARS-CoV-2 S₁ RBD-His protein if the antibody can bind to the SARS-CoV-2 S₁ RBD-His protein. This mixture (including, if one formed, an antibody-SARS-CoV-2 S₁ RBD-His protein complex) is added to a GFP-labeled HEK transfectant cell stably over-expressing human ACE-2 (hACE-2 HEK/eGFP Tfx). Binding between the SARS-CoV-2 S₁ RBD-His protein and the human ACE-2 is detected using an anti-His antibody. Exemplary results are shown in FIG. 2B-FIG. 2E. In the absence of a SARS-CoV-2 protein, no anti-His fluorescence is detected by flow cytometry (FIG. 2B); however, when a SARS-CoV-2 S₁ RBD protein is added to hACE-2 HEK/eGFP cells, a high level of anti-His fluorescence is detected (FIG. 2C). In the presence of irreverent or isotype control antibodies SARS-CoV-2 S₁/RBD proteins bind to ACE-2, and fluorescence remains high (FIG. 2D). In contrast, when anti-SARS-CoV-2 S₁ RBD antibodies are added, the antibodies form a complex with SARS-CoV-2 S₁/RBD proteins, preventing the viral proteins from binding ACE-2, resulting in decreased anti-His fluorescence (FIG. 2E).

FIGS. 3A and 3B. SARS-CoV-2 S₁ (100 ng/mL) (FIG. 3A) or SARS-CoV-2 S1 RBD (50 ng/mL) (FIG. 3B) His-tagged protein was incubated with hACE-2/eGFP cells in the absence of antibody (left panels), or in the presence of 25 μg/mL llama IgG control antibody, Recombinant LLAMABODY VHH antibody (70009-1). Protein binding was detected with fluorescently labeled anti-His monoclonal antibody and analyzed by flow cytometry. When a SARS-CoV-2 S₁ or RBD protein is added to hACE-2 HEK/eGFP cells, a high level of anti-His fluorescence is detected (left panels). In the presence of irrelevant or isotype control antibodies SARS-CoV-2 S₁/RBD proteins bind to ACE-2, and fluorescence remains high (llama IgG). In contrast, when anti-SARS-CoV-2 S₁ RBD antibody is added, the antibodies form a complex with SARS-CoV-2 S₁/RBD proteins, preventing the viral proteins from binding ACE-2, resulting in decreased anti-His fluorescence (70009-1).

FIG. 4. SARS-CoV-2 Active Trimer (500 ng/mL) His-tagged protein was incubated with hACE-2/eGFP cells in the absence of antibody (left panels), or in the presence of 25 μg/mL llama IgG control antibody or Recombinant LLAMABODY VHH antibody (70009-1). Protein binding was detected with fluorescently labeled anti-His monoclonal antibody and analyzed by flow cytometry. Anti-SARS-CoV-2 S₁ RBD antibody 70009-1 blocks binding of SARS-CoV-2 Active Trimer to hACE-2/eGFP cells.

FIGS. 5A and 5B. SARS-CoV-2 N439K variant (50 ng/mL) (FIG. 5A) or SARS-CoV-2 D614G variant (100 ng/mL) (FIG. 5B) His-tagged protein was incubated with hACE-2/eGFP cells in the absence of antibody (left panels), or in the presence of 25 μg/mL llama IgG control antibody or Recombinant LLAMABODY VHH antibody (70009-1). Protein binding was detected with fluorescently labeled anti-His monoclonal antibody and analyzed by flow cytometry. Anti-SARS-CoV-2 S₁ RBD antibody 70009-1 blocks binding of SARS-CoV-2 N439K and D614G variants to hACE-2/eGFP cells.

FIGS. 6A and 6B. SARS-CoV-2 London variant (500 ng/mL) (FIG. 6A) or SARS-CoV-2 P.1 variant (50 ng/mL) (FIG. 6B) His-tagged protein was incubated with hACE-2/eGFP cells in the absence of antibody (left panels), or in the presence of 25 μg/mL llama IgG control antibody or Recombinant LLAMABODY VHH antibody (70009-1). Protein binding was detected with fluorescently labeled anti-His monoclonal antibody and analyzed by flow cytometry. Anti-SARS-CoV-2 S₁ RBD antibody 70009-1 blocks binding of SARS-CoV-2 London and P.1 variants to hACE-2/eGFP cells.

FIGS. 7A and 7B. SARS-CoV S1 (100 ng/mL) (FIG. 7A) or SARS-CoV S1 RBD (50 ng/mL) (FIG. 7B) His-tagged protein was incubated with hACE-2/eGFP cells in the absence of antibody (left panels), or in the presence of 25 μg/mL llama IgG control antibody or Recombinant LLAMABODY VHH antibody (70009-1) to assess whether these antibodies could cross react with SARS-Cov. Protein binding was detected with fluorescently labeled anti-His monoclonal antibody and analyzed by flow cytometry. Anti-SARS-CoV-2 S₁ RBD antibody 70009-1 blocks binding of SARS-CoV S1 and S1 RBD to hACE-2/eGFP cells.

FIGS. 8A and 8B. MERS S1 (1 μg/mL) (FIG. 8A) or MERS S1 RBD (1 μg/mL) (FIG. 8B) Fc-tagged protein was incubated with hCD26/eGFP cells in the absence of antibody (left panels), or in the presence of 25 μg/mL llama IgG control antibody or Recombinant LLAMABODY VHH antibody (70009-1) to assess whether these antibodies could cross react with MERS. Protein binding was detected with fluorescently labeled anti-Fc monoclonal antibody and analyzed by flow cytometry. Anti-SARS-CoV-2 S₁ RBD antibody 70009-1 blocks binding of MERS S1 but not S1 RBD to hCD26/eGFP cells.

FIGS. 9A and 9B. Clustal Identity Matrix for 70009-1, 70009-2, and VHH-72. FIG. 9A shows ClustalW % Identity to 70009-1. FIG. 9B shows the alignment of 70009-1, 70009-2, and VHH-72 variable region amino acid sequences.

### DETAILED DESCRIPTION OF ILLUSTRATIVE EMBODIMENTS

This disclosure describes anti-SARS-CoV-specific antibodies, methods of making and characterizing those antibodies, and methods of using those antibodies. In some embodiments, the antibodies may bind to both SARS-CoV-1 and SARS-CoV-2. In some embodiments, the antibodies bind to S₁ of the spike protein of SARS-CoV-2 including, for example, to the receptor binding domain (RBD) of S₁. In some embodiments, the antibodies may block the binding of SARS-CoV-1 and/or SARS-CoV-2 to ACE-2.

### SARS-CoV-1 and SARS-CoV-2

Severe acute respiratory syndrome-coronavirus 2 (SARS-CoV-2) virus is a positive stranded RNA virus that is a member of the coronaviridae family, and is the causative agent of the COVID-19 pandemic (Lu et al. Lancet 395, 565-574 (2020), Wu et al. Nature 579, 265-269 (2020), Zhou et al. Nature 579, 270-273 (2020), Zhu et al. N Engl.J Med 382, 727-733 (2020)). Coronaviruses include four structural proteins: a spike protein (S), a membrane protein (M), an envelope protein (E), and a nucleocapsid protein (N). The spike protein is composed of two subunits: S₁ which is responsible for binding to the host cell receptor via a highly conserved receptor binding domain (RBD) (Lan et al. Nature 581, 215-220 (2020)), and S2 which facilitates fusion of the virus to a target cell membrane.

Like the related severe acute respiratory syndrome (SARS) virus, SARS-CoV-1, the causative agent of the 2002 SARS pandemic, SARS-CoV-2 primarily infects cells of a host's respiratory system by binding to the angiotensin converting enzyme 2 (ACE-2) (Chen et al. Biochem Biophys Res Commun, (2020), Letko et al. Nat Microbiol 5, 562-569 (2020), Li et al. Nature 426, 450-454 (2003), Walls et al. Cell 181, 281-292 e286 (2020)) which is expressed in vascular endothelia. Other organ systems are also involved, however, due to the expression of ACE-2 on cells in cardiovascular tissue, kidney, and bladder tissue, and on the epithelia of the small intestine and testes (Zou et al. Front Med 14, 185-192 (2020)). The resulting inflammation and tissue damage caused either by direct viral infection by vascular epithelial cells, or indirectly by host immune responses to infection and subsequent cytokine storm, can, in cases of severe, result in severe respiratory failure with multiple organ failure and death (Yuki et al. Clin Immunol 215, 108427 (2020)).

Phylogenetically, SARS-CoV-2 and SARS-CoV-1 belong to the Betacoronavirus genus which can be found in human and many animal species (Lu et al. Lancet 395, 565-574 (2020), Wu et al. Nature 579, 265-269 (2020), Zhou et al. Nature 579, 270-273 (2020), Zhu et al. N Engl J Med 382, 727-733 (2020)). Although both viruses have been responsible for independent severe acute respiratory syndromes in humans, SARS-CoV-2 is more closely related to bat coronavirus RaTG13, sharing more than 93% homology in the spike gene, than SARS-CoV-1 (Zhou et al. Nature 579, 270-273 (2020)). Sequence analysis of the S gene of SARS-CoV-2 and SARS-CoV-1 reveals that spike proteins of the two viruses are highly divergent, sharing about 76% homology while the receptor binding protein shares less than 75% homology (Zhou et al. Nature 579, 270-273 (2020), Ou et al. Nat Commun 11, 1620 (2020)); still, the portion of the receptor binding domains for both viruses that is critical for binding to ACE-2 is nearly identical (Lan et al. Nature 581, 215-220 (2020)) suggesting that antibodies that block the correct portion of the receptor binding domain of SARS-CoV-2 may also block the receptor binding domain of SARS-CoV-1.

A third member of the Betacoronavirus genus, Middle East respiratory syndrome coronavirus (MERS-CoV), was identified as the causative agent of a human severe respiratory syndrome in 2012 in Saudi Arabia (Zaki et al. N Engl J Med 367, 1814-1820 (2012)), known as Middle East respiratory syndrome (MERS). Whereas SARS-CoV-1 and SARS-CoV-2 use human ACE-2 as their main receptor, MERS-CoV uses transmembrane dipeptidylpeptidase 4 (DPP4), also known as CD26, as its primary receptor. The homology between the receptor binding domains (RBDs) of SARS-CoV-2 spike protein and MERS-CoV spike protein is low, only 19.1%; however, both SARS-CoV-2 and MERS-CoV can bind DPP4 by virtue of identical amino acids at their respective DPP4 binding residues (Li et al. iScience 23, 101160 (2020)).

Commercial and government vaccine programs have recently succeeded in obtaining FDA approval for several SARS-COV-2 vaccines, and world-wide vaccination programs are underway. However, there is great concern about the emergence of several viral variants. The FDA has granted emergency use authorization for the broad-spectrum antiviral drug remdesivir to treat COVID-19 based on studies suggesting efficacy of the drug against SARS and MERS coronaviruses (Beigel et al. N Engl J Med, (2020), de Wit et al. Proc Natl Acad Sci USA 117, 6771-6776 (2020)).

The FDA has also granted emergency use authorization for convalescent plasma therapy to treat COVID-19. COVID-19 convalescent plasma contains polyclonal immunoglobulin (IgG) with the potential to bind to the SARS-CoV-2 virus and neutralize the ability of the virus to infect host cells. While this type of therapy has been used successfully with other viruses (Sullivan et al. Transfus Med Rev 34, 145-150 (2020)), it can also be problematic because the specificities of the anti-viral IgG present in convalescent plasma is unknown. For example, effective anti-SARS-CoV-2 immune responses may generate antibodies that binds to many different SARS-CoV-2 proteins (including, for example, the N, M, E and S proteins), but only a small fraction of those antibodies may be capable of binding to the specific S₁ and RBD protein sequences required by SARS-CoV-2 to enter host cells.

A more effective SARS-CoV-2 therapy may include administration of high titers of a monoclonal antibody designed to specifically bind either the host ACE-2 enzyme or SARS-CoV-2 spike protein (for example, S₁ and/or the RBD) that has been shown to prevent the ability of the SARS-CoV-2 to bind to the host ACE-2 enzyme. Further therapies may include the administration of a cocktail of two, three, four, five, or more monoclonal antibodies of differing specificities and origin.

### Camelid Antibodies

The canonical view of antibodies is of molecules composed of two heavy chains and two light chains. An intact human antibody molecule has two heavy (H) chain variable regions (abbreviated herein as VH or V_{H}) and two light (L) chain variable regions (abbreviated herein as VL or V_{L}). The V_{H} and V_{L} regions can be further subdivided into regions of hypervariability, termed “complementarity determining regions” (“CDRs”), interspersed with regions that are more conserved, termed “framework regions” (“FRs”). The extent of the FRs and CDRs has been precisely defined (see, Kabat et al. (1991) Sequences of Proteins of Immunological Interest, Fifth Edition, U.S. Department of Health and Human Services, NIH Publication No. 91-3242, and (Chothia et al. J Mol Biol 196, 901-917 (1987))). Each VH and VL is composed of three CDRs and four FRs, arranged from amino-terminus to carboxy-terminus in the following order: FR1, CDR1, FR2, CDR2, FR3, CDR3, FR4.

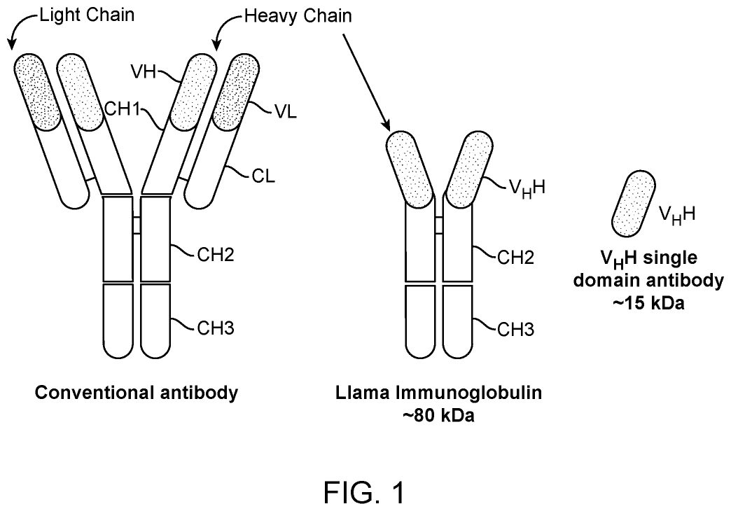

However, mammals from the Camelidae family that include llamas, camels, and alpacas produce two types of antibodies. One type of antibody camelids produce is the conventional antibody made up of two heavy chains and two light chains. In addition, they produce another type of antibody that is composed of two identical heavy-chain polypeptides, each of which incorporates contiguous constant domains, a hinge region, and a variable domain. This unique secondary set of single-chain antibodies (scAbs), also known as heavy chain IgG (hcIgG) or heavy chain-only antibodies (HCAbs), contain a single variable domain antigen binding domain, called the VHH region or VHH region, instead of two variable domains (VH and VL) that make up the equivalent antigen-binding fragment (Fab) of conventional IgG antibodies. FIG. 1 shows the structure of human immunoglobulin, llama immunoglobulin, and a VHH single domain antibody.

Each variable domain of the camelid scAb can function independently as an antigen-binding module. This single variable domain, in the absence of an effector domain, is referred to as a single-domain antibody (sdAb) (also referred to as VHH antibody, VHH antibody, VHH, VHH, LLAMABODY™, or NANOBODY®) and has a length of are about 110 amino acids and a molecular weight of only 12-15 kDa, compared to 150-160 kDa for a conventional antibody. The term VHH was originally introduced to indicate a VH domain derived from camelid heavy chain antibodies. The lack of a light chain does not limit or reduce the diversity of the epitopes recognized or antigen binding and VHHs demonstrate affinities and specificities for antigens comparable to conventional antibodies.

More information on camelid single-chain antibodies and single-domain antibodies can be found, for example, at rndsystems.com/products/llamabody-camelid-antibodies; Wrapp et al., Cell. 2020 Jun. 11; 181(6):1436-1441. doi: 10.1016/j.cell.2020.05.047; Arbabi-Ghahroudi, Front Immunol. 2017 Nov. 20; 8:1589. doi: 10.3389/fimmu.2017.01589; Desmyter et al., Curr Opin Struct Biol. 2015 June; 32:1-8. doi: 10.1016/j.sbi.2015.01.001; Fernandes et al., Frontlmmunol. 2017 Jun. 9; 8:653. doi: 10.3389/fimmu.2017.00653; and WO2002085944.

VHH single domain antibodies provide many benefits over traditional IgG antibodies. Due to their smaller size, they are able to detect epitopes that may not have been accessible with a traditional antibody due to steric hindrance. They are able to penetrate tissue and enter cells more easily, allowing for more specific IHC staining and intracellular flow cytometry staining. VHH single domain antibodies demonstrate improved thermal stability and chemostability compared to conventional most antibodies, withstanding larger pH and temperature ranges. Unlike conventional antibodies they are functional at high temperatures and refold after heat denaturation and demonstrate improved stability after prolonged storage. These key characteristics, including, but not limited to, high affinity and specificity (equivalent to conventional antibodies), high thermostability, good solubility, monomeric behavior, small size, relatively low production cost, ease of genetic engineering, format flexibility or modularity, low immunogenicity, and a higher penetration rate into tissues make VHHs ideal for biotechnological and medical applications.

For example, the use of VHHs as biologics against respiratory pathogens, such as respiratory syncytial virus (RSV), is an attractive application, since the highly stable VHHs can be nebulized and administered via an inhaler directly to the site of infection (Respaud et al., Expert Opin Drug Deliv. 2015 June; 12(6):1027-39. doi: 10.1517/17425247.2015.999039; Van Heeke et al., Pharmacol Ther. 2017 January; 169:47-56. doi: 10.1016/j.pharmthera.2016.06.012; and Detalle et al., Antimicrob Agents Chemother. 2015 Oct. 5; 60(1):6-13. doi: 10.1128/AAC.01802-15). single domain antibody humanization strategy.

For therapeutic applications, the immunogenicity of VHHs can be reduced via humanization, following, for example the methods described in more detail by Vincke et al. (Vincke et al, J Biol Chem. 2009 Jan. 30; 284(5):3273-3284. doi: 10.1074/jbc.M8068892002009). VHH antibodies can be recombinantly expressed at high levels in prokaryotic (Escherichia coli) and eukaryotic cells (yeast).

### Anti-SARS-CoV Antibodies

In one aspect, this disclosure describes an antibody that binds to SARS-CoV (that is, an anti-SARS CoV antibody). An antibody that binds to SARS-CoV includes an antibody that binds to SARS-CoV-1 and/or SARS-CoV-2. In some embodiments, an antibody that binds to SARS-CoV may bind to both SARS-CoV-1 and SARS-CoV-2. In some embodiments, an antibody that binds to SARS-CoV may bind to SARS-CoV-1 but not SARS-CoV-2 or to SARS-CoV-2 but not SARS-CoV-1. In some embodiments an anti-SARS-CoV antibody preferably binds to SARS-CoV-2 and may optionally bind to SARS-CoV-1.

In some embodiments, an antibody that binds to SARS-CoV-2 may also inhibit the binding of SARS-CoV-1 S₁ to ACE-2 or may inhibit the binding of SARS-CoV-1 S₁ RBD to ACE-2. For example, as described in Example 1, the llama-derived VHH antibody 70009-1 inhibited SARS-CoV-1 S₁ and SARS-CoV-1 S₁ RBD binding to ACE-2 as well as SARS-CoV-2 S₁ and SARS-CoV-2 S₁ RBD binding to ACE-2.

In some embodiments, the antibody may decrease the binding of SARS-CoV (including SARS-CoV-1, SARS-CoV-2, SARS-CoV-1 spike protein, SARS-CoV-1 S₁, SARS-CoV-1 S₁ RBD, SARS-CoV-2 spike protein, SARS-CoV-2 S₁, and/or SARS-CoV-2 S₁ RBD) to a SARS-CoV ligand (including, for example, ACE-2) by at least 10 percent (%), at least 15%, at least 20%, at least 25%, at least 30%, at least 40%, at least 50%, at least 60%, at least 70%, at least 80%, at least 90%, at least 95%, or at least 98%. In some embodiments, the antibody may decrease the binding of SARS-CoV to a SARS-CoV ligand (including, for example, ACE-2) by up to 99% or up to 99.5%. For example, the antibody may decrease the binding of SARS-CoV to a SARS-CoV ligand by 50% to 99.5% or by 80% to 99.5% In some embodiments, the binding of SARS-CoV to a SARS-CoV ligand may be measured using an antibody-blocking assay as described in the Examples.

In some embodiments, an antibody that binds to SARS-CoV may inhibit the binding of SARS-CoV-2 S₁ to ACE-2 and/or may inhibit the binding of SARS-CoV-2 S₁ RBD to ACE-2. For example, as described in Example 1, the llama-derived single domain VHH antibody 70009-1 reduced the binding of SARS-CoV-2 S₁ RBD to ACE-2 expressing cells.

In some embodiments, the antibodies may bind to a SARS-CoV-2 variant, for example, binding to the S₁ of the spike protein of a variant SARS-CoV-2 including, for example, to the receptor binding domain (RBD) of S₁. In some embodiments, the antibodies may block the binding of a variant SARS-CoV-2 to ACE-2. Across the world, multiple variants of the severe acute respiratory syndrome coronavirus 2 (SARS-CoV-2) have emerged. Three new variants have rapidly become dominant within their countries and have aroused concerns: B.1.1.7 (also known as VOC-202012/01), 501Y.V2 (B.1.351), and P.1 (B.1.1.28.1). The B.1.17 variant (also referred to herein as the London variant) has 23 mutations with 17 amino acid changes and was first described in the United Kingdom on Dec. 14, 2020. One significant mutation is an N501Y mutation in the spike protein that increases the binding affinity of the virus to the ACE2 receptor. Another mutation, a D614G mutation, makes the virus more transmissible. The Brazilian SARS-CoV-2 P.1 variant (also referred to herein as the P.1 variant) was first reported in Brazil on Jan. 12, 2021. It has approximately 35 mutations with 17 amino acid changes, including the N501Y mutation in the spike protein which increases binding affinity to the ACE2 receptor. The P.1 is resistant to several neutralizing monoclonal antibodies, over six times more resistant to neutralization by convalescent plasma, and more than twice as resistant to sera from vaccinees than the wildtype virus. See, for example, Galloway et al., MMWR Morb Mortal Wkly Rep. 2021 Jan. 22; 70(3): 95-99. doi: 10.15585/mmwr.mm7003e2; Karim and Oliveira, “New SARS-CoV-2 Variants—Clinical, Public Health, and Vaccine Implications,” NEJM, Mar. 24, 2021, doi: 10.1056/NEJMc2100362; Public Health England. Investigation of novel SARS-CoV-2 variant: variant of concern 202012/01, technical briefing 3. London, United Kingdom: Public Health England; 2020; Faria et al., (12 Jan. 2021). “Genomic characterisation of an emergent SARS-CoV-2 lineage in Manaus: preliminary findings,” Virological; and Covid-19 Genomics UK Consortium (15 Jan. 2021). “COG-UK Report on SARS-CoV-2 Spike mutations of interest in the UK” (PDF) (available on the worldwide web at cogconsortium.uk).

In some embodiments, the antibody includes a heavy chain variable region with an amino acid sequence that is at least 70%, at least 80%, at least 85%, at least 90%, at least 95%, at least 96%, at least 97%, at least 98%, or at least 99% identical to an amino acid sequence of the VHH domain of the 70009-1 antibody or the 70009-2 antibody. In some embodiments, the antibody includes the amino acid sequence of the single domain VHH domain of 70009-1 or 70009-2.

As described in Examples 1 and 2, the amino acid sequences of the 70009-1 and 70009-2 single domain VHHs were obtained from B lymphocytes obtained from a llama immunized with amino acids 319-541 of SARS-CoV-2 S₁. The amino acid sequences of the 70009-1 and 70009-2 VHHs are shown below.

70009-1
(SEQ ID NO: 1)
QLQLVESGGGLVQPGGSLRLSCAASTLTLNSYAIGWFRQAPGKEREAVS
CTSSSGDMTYYANSVKGRFTISRDNAKNTVYLQMSRLKPEDTAVYYCAA
VKLEYGYVCSHPNEYDYWGQGTQVTVSS
70009-2
(SEQ ID NO: 2)
QLQLVESGGGLVQPGGSLRLSCAASTLTLNSYAIGWFRQAPGKEREAVS
CTSSSGDMTYYTNSVKGRFTISRDNAKNTVYLQMSRLKPDDTAVYYCAA
VLLEYGYVCSHPNEYDYWGQGTQVTVSS

In some embodiments, the antibody includes a variable region with an amino acid sequence that can contain one, two, three, four, five, six, or more amino acid substitutions in the variable domain amino acid sequence of 70009-1 (SEQ ID NO: 1) or 70009-2 (SEQ ID NO: 2). In some embodiments, the substitutions may be substitutions with conserved amino acids. In some embodiments, the amino acid substitutions do not substantially affect binding of the antibody to SARS-CoV, and, in some embodiments, preferably do not substantially affect binding of the antibody to SARS-CoV-2.

In some embodiments, an antibody includes one or more, at least one, at least two, or three complementary determining regions (CDRs) selected from:
a CDR1 sequence comprising an amino acid sequence which is identical to, or differs by one, two, or three amino acids from, SYAIG (SEQ ID NO: 3);
a CDR2 sequence comprising an amino acid sequence which is identical to, or differs by one, two, three, four, or five amino acids from, CTSSSGDMTYYANSVKG (SEQ ID NO: 4) or CTSSSGDMTYYTNSVKG (SEQ ID NO: 6);
a CDR3 sequence comprising an amino acid sequence which is identical to, or differs by one, two, three, four, or five amino acids from, VKLEYGYVCSHPNEYDY (SEQ ID NO: 5) or VLLEYGYVCSHPNEYDY (SEQ ID NO: 7).

In some embodiments, the substitutions may be substitutions with conserved amino acids. In some embodiments, the amino acid substitutions do not substantially affect binding of the antibody to SARS-CoV, and, in some embodiments, preferably do not substantially affect binding of the antibody to SARS-CoV-2.

In some embodiments, an antibody includes all three CDR regions of the 70009-1 VHH, with a CDR1 amino acid sequence including SYAIG (SEQ ID NO: 3), a CDR2 amino acid sequence including CTSSSGDMTYYANSVKG (SEQ ID NO: 4), and a CDR3 amino acid sequence including VKLEYGYVCSHPNEYDY (SEQ ID NO: 5).

In some embodiments, an antibody includes all three CDR regions of the 70009-2 VHH, with a CDR1 amino acid sequence including SYAIG (SEQ ID NO: 3), a CDR2 amino acid sequence including CTSSSGDMTYYTNSVKG (SEQ ID NO: 6), and a CDR3 amino acid sequence including VLLEYGYVCSHPNEYDY (SEQ ID NO: 7).

In some embodiments, an antibody can contain one, two, three, four, five, six, or more amino acid substitutions in one or more framework regions (FRs). In some embodiments, the substitutions in the framework regions (FRs) do not substantially affect binding of the antibody to SARS-CoV, and, in some embodiments, preferably do not substantially affect binding of the antibody to SARS-CoV-2. In some embodiments, the substitutions in one or more FRs may be substitutions with conserved amino acids.

In some embodiments, the anti-SARS CoV antibody is preferably a monoclonal antibody.

In some embodiments, an anti-SARS-CoV antibody includes an antibody that binds to the same SARS-CoV epitope as a VHH having the amino acid sequence of 70009-1 (SEQ ID NO: 1) or 70009-2 (SEQ ID NO: 2).

In some embodiments, the antibody may be an isolated antibody. In some embodiments, the antibodies may be isolated or purified by conventional immunoglobulin purification procedures, such as protein A- or G-Sepharose, hydroxylapatite chromatography, gel electrophoresis, dialysis, or affinity chromatography.

In some embodiments, an antibody that binds to SARS-CoV may include a derivative of an antibody that is modified or conjugated by the covalent attachment of any type of molecule to the antibody. Such antibody derivatives include, for example, antibodies that have been modified by glycosylation, acetylation, pegylation, phosphorylation, amidation, derivatization by known protecting/blocking groups, proteolytic cleavage, toxins, or linkage to a cellular ligand or other protein. Any of numerous chemical modifications may be carried out by known techniques, including, but not limited to, specific chemical cleavage, acetylation, formylation, and metabolic synthesis of tunicamycin. Additionally, the derivatives may contain one or more non-classical amino acids.

An antibody that binds to SARS-CoV may be coupled directly or indirectly to a detectable marker by techniques well known in the art. A detectable marker is an agent detectable, for example, by spectroscopic, photochemical, biochemical, immunochemical, or chemical means. Useful detectable markers include, but are not limited to, fluorescent dyes, chemiluminescent compounds, radioisotopes, electron-dense reagents, enzymes, coenzymes, colored particles, biotin, or dioxigenin. A detectable marker often generates a measurable signal, such as radioactivity, fluorescent light, color, or enzyme activity. Antibodies conjugated to detectable agents may be used for diagnostic or therapeutic purposes. Examples of detectable agents include various enzymes, prosthetic groups, fluorescent materials, luminescent materials, bioluminescent materials, radioactive materials, positron emitting metals using various positron emission tomographies, and nonradioactive paramagnetic metal ions. The detectable substance may be coupled or conjugated either directly to the antibody or indirectly, through an intermediate such as, for example, a linker known in the art, using techniques known in the art. See, for example, U.S. Pat. No. 4,741,900, describing the conjugation of metal ions to antibodies for diagnostic use. Examples of suitable enzymes include horseradish peroxidase, alkaline phosphatase, beta-galactosidase, and acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride and phycoerythrin; an example of a luminescent material includes luminol; examples of bioluminescent materials include luciferin, and aequorin; and examples of suitable radioactive material include iodine (¹²¹I, ¹²³I, ¹²⁵I, ¹³¹I), carbon (¹⁴C), sulfur (³⁵S), tritium (³H), indium (¹¹¹In, ¹¹²In, ¹¹³mIn, ¹¹⁵mIn), technetium (⁹⁹Tc, ⁹⁹mTc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y ⁴⁷Sc, ¹⁸⁶Re, ¹⁸⁸Re, ¹⁴²Pr, ¹⁰⁵Rh, and ⁹⁷Ru. Techniques for conjugating such therapeutic moieties to antibodies are well-known.

An antibody that binds to SARS-CoV may be part of an Antibody Drug Conjugate (ADC), conjugated to a payload. In the components of an ADC (the payload, linker, and antibody), payload a crucial part with cytotoxic potency. Payloads for ADCs can be small molecules (cellular toxins), protein toxins, proteins, enzymes, and radionuclides. Specific payloads include, but are not limited to ozogamicin, vedotin, emtansine, pasutotox, deruxtecan, auristatins, monomethyl auristatin E, monomethyl auristatin F, maytansinoid DM1, maytansinoid DM4, doxorubicin, mytansinoids, tubulysins, crptophycins, hemiasterlin, cemadotin, rhizoxin, discodermolide, pyrrolobenzodiazepines, duocarmycins, calicheamicins, camptothecin, irinotecan, topotecan, indolinobenzodiazepine, apoptosis inducers, spliceosome targets, transcription inhibitors, translation inhibitors, cytokines, and chemokines.

The antibody may include sequences from antibodies for other suitable species. For example, the antibody may include sequences from a human antibody, a mouse antibody, a rat antibody, a rabbit antibody, a goat antibody, a shark antibody, a llama antibody, etc. In some embodiments, one or more of the variable regions and/or CDR regions of the antibody may include an antibody sequence (e.g., a constant region) of any suitable species (e.g., rat, rabbit, goat, shark, llama, etc.).

In some embodiments, an antibody that binds to SARS-CoV may include at least a portion of an immunoglobulin constant region (Fc) of a human immunoglobulin, for example, the Fc region of a human IgG, IgE, IgM, or IgD antibody. In some embodiments, the human Fc region may be of the IgG1, IgG2, IgG3, IgG4, IgA1 or IgA2 subclass. In some embodiments, a VHH antibody is fused to a human Fc IgG2 region. In some embodiments, a VHH antibody is fused to a full or a portion of a murine IgG constant region of any isotype subclass. In some embodiments, a VHH antibody is fused to a full or a portion of a goat, rabbit, chicken rat, or hamster IgG constant region of any isotype subclass.

The antibody may be of any type, any class, or any subclass. When the antibody is a human or mouse antibody, for example, the type may include, for example, IgG, IgE, IgM, IgD, IgA and IgY; and/or the class may include, for example, IgG1, IgG2, IgG3, IgG4, IgA1 and IgA2.

In some embodiments, the antibody is an IgG antibody. In some embodiments, the IgG antibody may be a human antibody of any one of the IgG subclasses including, for example, IgG1, IgG2, IgG3 or IgG4. In some embodiments, the antibody may be a mouse IgG of one of the following sub-classes: IgG1, IgG2A, IgG2B, IgG2C and IgG3. In some embodiments, the antibody may be a rat IgG of one of the following sub-classes: IgG1, IgG2A, IgG2B, or IgG2C.

In some embodiments, the antibody may be paired with a light chain, for example, a human kappa light chain or human lambda light chain.

In some embodiments, the antibody includes an antibody fragment capable of binding to a biological molecule (such as an antigen or receptor) or portions thereof, including but not limited to Fab, Fab′ and F(ab′)2, pFc′, Fd, a single domain antibody (sdAb), a variable fragment (Fv), a single-chain variable fragment (scFv) or a disulfide-linked Fv (sdFv); a diabody or a bivalent diabody; a linear antibody; a single-chain antibody molecule; and a multispecific antibody formed from antibody fragments.

In some embodiments, the antibody may be a humanized antibody. An antibody that binds to SARS-CoV may be humanized by any suitable method. For example, humanization of the antibody may include changes to the antibody to reduce the immunogenicity of the antibody when used in humans. In some embodiments, a humanized antibody that binds to SARS-CoV may include at least a portion of an immunoglobulin constant region (Fc) of a human immunoglobulin. Techniques for producing humanized monoclonal antibodies may be found, for example, in Jones et al. (Jones et al. Nature 321, 522-525 (1986)) and Singer et al. (Singer et al. J Immunol 150, 2844-2857 (1993)). Techniques for humanized camelid-derived monoclonal antibodies may be found, for example, in Vincke (Vincke et al, J Biol Chem. 2009 Jan. 30; 284(5):3273-3284. doi: 10.1074/jbc.M8068892002009).

In some embodiments, a monoclonal antibody includes a chimeric antibody, that is, an antibody in which different portions are derived from different animal species. A chimeric antibody may be obtained by, for example, splicing the genes from a mouse antibody molecule with appropriate antigen specificity together with genes from a human antibody molecule of appropriate biological specificity. See, for example, Takeda et al. (Takeda et al. Nature 314, 452-454 (1985)). Additional chimeric antibodies including genes from different species may be envisioned.

VHHs can be constructed into a variety of formats, including, but not limited to, bivalent, multivalent, bispecific, and multispecific formats. The term bispecific antibody (bsAb) is used to describe an antibody molecule that can simultaneously bind to two different epitopes or antigens. A bispecific antibody includes two variable regions with differing antigen specificities. A multispecific antibody includes more than one variable region of differing antigen specificities, for example, two, three, four, or more variable regions. A bivalent antibody has at least two antigen-binding sites and a multivalent antibody binds to multiple sites on one target. In some embodiments, an antibody includes a bivalent antibody that includes more than one variable region targeting a similar molecule. In other embodiments, an antibody includes a multivalent antibody that comprises more than one variable region targeting a similar molecule. Bivalent, multivalent, bispecific and multispecific antibodies, including such llama antibodies, are described in more detail in, for example, Strokappe et al., 2019, Antibodies (Basel); 8(2):38; Beirnaert et al., 2017, Front Immunol; 8:867; Li et al., 2020, Clin Transl Med; 9(1):16; Coppieters et al., 2006, Arthritis Rheum; 54(6):1856-66; Weiss and Verrips, 2019, Vaccines (Basel); 7(3):77; Sadeghi et al., 2020, Drug Test Anal; 12(1):92-100; Hultberg et al., 2011, PLoS One; 6(4):e17665; Zhang and Mackenzie, 2012, Methods Mol Biol; 911:445-56; Stone et al., 2007, J Immunol Methods; 318(1-2):88-94; Dong et al., 202, Sci Rep; 10(1):17806; Godar et al., 2018, J Allergy Clin Immunol; 142(4):1185-1193.e4; Rozan et al., 2013, Mol Cancer Ther; 12(8):1481-91; Els Conrath et al., 2001, J Biol Chem; 276(10):7346-50; Palomo et al., 2016, Antimicrob Agents Chemother; 60(11):6498-6509; and Labrijn et al., 2019, Nature Reviews Drug Discovery; 18:585-608.

VHH antibodies can be fused to wide variety of molecules. For example, VHHs can be formatted to serve as a targeting moiety for an effector function such as a tubulin inhibitor molecule, a toxin, an antiviral drug, or an antibody-derived Fc domain. (De Vlieger et al., Antibodies (Basel). 2018 Dec. 20; 8(1):1. doi: 10.3390/antib8010001).

In some embodiments, the antibody may be produced by an animal (including, but not limited to, human, mouse, rat, rabbit, hamster, goat, horse, chicken, or turkey), produced by a cell from an animal, chemically synthesized, or recombinantly expressed. The antibody may be purified by any method known in the art for purification of an immunoglobulin molecule, for example, by chromatography (for example, ion exchange, affinity, and sizing column chromatography), centrifugation, differential solubility, or by any other standard technique for the purification of proteins. In addition, an antibody may be fused to a heterologous polypeptide sequence, as described herein or otherwise known in the art, including, for example, to facilitate purification.

In some embodiments, an antibody that binds to SARS-CoV may be made by immunizing an animal with a SARS-CoV protein or fragment thereof (including, for example, SARS-CoV-1 spike protein, SARS-CoV-1 S₁, SARS-CoV-1 S₁ RBD, SARS-CoV-2 spike protein, SARS-CoV-2 S₁, and/or SARS-CoV-2 S₁ RBD). In some embodiments, an antibody that binds to SARS-CoV may be made by immunizing an animal with at least a portion of SARS-CoV-2 S₁ (UniProt PODTC2). In some embodiments, the animal may be a mammal. For example, the animal may be a rabbit, a mouse, a goat, a sheet, a llama, or a rat. In some embodiments, the animal may be a chicken.

A monoclonal antibody may be assayed for immunospecific binding by the methods described herein and by any suitable method known in the art. The immunoassay that may be used includes but is not limited to a competitive and/or a non-competitive assay system using a technique such as BIACORE analysis, fluorescence activated cell sorter (FACS) analysis, immunofluorescence, immunocytochemistry, Western blot, radio-immunoassay, enzyme linked immunosorbent assay (ELISA), “sandwich” immunoassay, immunoprecipitation assay, precipitin reaction, gel diffusion precipitin reaction, immunodiffusion assay, agglutination assay, complement-fixation assay, immunoradiometric assay, fluorescent immunoassay, and protein A immunoassay. Such assays are routine and well known in the art (see for example, Ausubel et al., eds, Current Protocols in Molecular Biology, Vol. 1, John Wiley & Sons, Inc., N.Y. (1994)).

A monoclonal antibody may be obtained by any suitable technique. In some embodiments, an antibody that binds to SARS-CoV may be made by recombinant DNA methods, produced by phage display, and/or produced by combinatorial methods. DNA encoding an antibody that binds to SARS-CoV may be readily isolated and sequenced using conventional procedures.

Once isolated, the DNA may be transfected into a host cell (including, for example, simian COS cells, Chinese hamster ovary (CHO) cells, human embryonic kidney cells (HEK), or myeloma cells that do not otherwise produce immunoglobulin protein) or introduced into a host cell by genome editing (for example, using a CRISPR-Cas system) to obtain the synthesis of monoclonal antibodies in a recombinant host cells. The DNA encoding an antibody that binds to SARS-CoV may be modified to, for example, humanize the antibody.

In another aspect, this disclosure describes an isolated polynucleotide molecule. In some embodiments, the isolated polynucleotide molecule includes a nucleotide sequence encoding an antibody. In some embodiments, the isolated polynucleotide molecule includes a nucleotide sequence that has at least 75%, at least 80%, at least 85%, at least 90%, at least 95%, at least 96%, at least 97%, at least 98%, or at least 99% sequence identity to nucleotide sequence encoding an antibody described herein. In some embodiments, the isolated polynucleotide molecule includes polynucleotides that hybridize under high stringency to a nucleotide sequence encoding an antibody or a complement thereof. As used herein “stringent conditions” refer to the ability of a first polynucleotide molecule to hybridize, and remain bound to, a second, filter-bound polynucleotide molecule in 0.5 M NaHPO₄, 7% sodium dodecyl sulfate (SDS), and 1 mM EDTA at 65° C., followed by washing in 0.2×SSC/0.1% SDS at 42° C. (see Ausubel et al. (eds.), Current Protocols in Molecular Biology, Vol. 1, Green Publishing Associates, Inc., and John Wiley & Sons, Inc., N.Y. (1989), at p. 2.10.3). In some embodiments, the isolated polynucleotide molecule includes polynucleotides that encode one or more of the CDRs or the variable region of an antibody of the present disclosure. General techniques for cloning and sequencing immunoglobulin variable domains and constant regions are well known. See, for example, Orlandi et al. (Orlandi et al. Proc Natl Acad Sci USA 86, 3833-3837 (1989)).

In another aspect, this disclosure describes recombinant vectors including an isolated polynucleotide of the present disclosure. The vector may be, for example, in the form of a plasmid, a viral particle, or a phage. The appropriate DNA sequence may be inserted into a vector by a variety of procedures. In general, the DNA sequence is inserted into an appropriate restriction endonuclease site(s) in a vector by procedures known in the art. Such procedures are deemed to be within the scope of those skilled in the art. Large numbers of suitable vectors and promoters are known to those of skill in the art and are commercially available. The following vectors are provided by way of example. Bacterial vectors include, for example, pQE70, pQE60, pQE-9, pBS, pD10, phagescript, psiX174, pbluescript SK, pbsks, pNH8A, pNH16a, pNH18A, pNH46A, ptrc99a, pKK223-3, pKK233-3, pDR540, and pRIT5. Eukaryotic vectors include, for example, pWLNEO, pSV2CAT, pOG44, pXT1, pSG, pSVK3, pBPV, pMSG, and pSVL. However, any other plasmid or vector may be used.

In a further aspect, this disclosure also includes a host cell containing at least one of the above-described vectors. The host cell may be a higher eukaryotic cell, such as a mammalian or insect cell, or a lower eukaryotic cell, such as a yeast cell. Or, the host cell may be a prokaryotic cell, such as a bacterial cell, or a plant cell. Introduction of a vector construct into the host cell may be effected by any suitable techniques, such as, for example, calcium phosphate transfection, DEAE-Dextran mediated transfection, electroporation, or nucleofection.

Antibodies of the present disclosure may be expressed in mammalian cells, yeast, bacteria, or other cells under the control of appropriate promoters. Cell-free translation systems may also be employed to produce such proteins using RNAs derived from the DNA constructs of the present disclosure. Appropriate cloning and expression vectors for use with prokaryotic and eukaryotic hosts are described by, for example, Sambrook et al., Molecular Cloning: A Laboratory Manual, Second Edition, Cold Spring Harbor, N.Y. (1989).

Also included in the present disclosure are phage display libraries expressing one or more hypervariable regions from an antibody of the present disclosure, and the clones obtained from such a phage display library. A phage display library is used to produce antibody-derived molecules. Gene segments encoding the antigen-binding variable domains of antibodies are fused to genes encoding the coat protein of a bacteriophage. Bacteriophage containing such gene fusions are used to infect bacteria, and the resulting phage particles have coats that express the antibody-fusion protein, with the antigen-binding domain displayed on the outside of the bacteriophage. Phage display libraries may be prepared, for example, using the PH.D.-7 Phage Display Peptide Library Kit (Catalog #E8100S) or the PH.D.-12 Phage Display Peptide Library Kit (Catalog #E8110S), available from New England Biolabs Inc., Ipswich, MA. See, for example, Smith and Petrenko (Smith et al. Chem Rev 97, 391-410 (1997)).

### Uses for the Anti-SARS-CoV Antibodies

An antibody that binds to SARS-CoV, as described herein, may be used for any suitable application. For example, a monoclonal antibody may be used in both in vitro and in vivo diagnostic and therapeutic methods.

In some embodiments, an antibody may be used to determine a level of expression of SARS-CoV protein in vitro or in vivo. In some embodiments, an antibody may be used to label a cell in vivo or in vitro. In some embodiments, an antibody may be used to determine a level of expression of SARS-CoV protein in a patient sample.

In some embodiments, an antibody may be used to identify the presence or absence of SARS-CoV protein in a sample from a subject. In some embodiments, identifying the presence of SARS-CoV may include identifying an amount of SARS-CoV in a sample from a subject.

The sample from the subject may include any suitable or useful samples. Exemplary samples include saliva, sputum, blood, urine, feces, nasal swabs, and bronchial brush or bronchoalveolar lavage (BAL) fluid.

In some embodiments, the antibody may be labeled. The antibodies may be labeled with one or more detectable markers, as described herein. For example, a labeled antibody may be used to label a cell, and the labeled cell may be directly or indirectly imaged via secondary methods. In some embodiments, the cell may be a mammalian cell.

In some embodiments, the antibody may be used to modulate the interaction of SARS-CoV and a ligand of SARS-CoV including, for example, ACE-2. In some embodiments, modulation of the interaction of SARS-CoV and ACE-2 can include inhibiting the interaction of SARS-CoV and ACE-2. Such inhibition may produce immunotherapeutic effects including, for example, the prevention of infection of a cell expressing ACE-2 by SARS-CoV, delaying the onset of SARS or COVID-19, and/or delaying the progression of SARS or COVID-19.

This disclosure further described a kit including an antibody. For example, a kit may include a composition that includes an anti-SARS-CoV monoclonal antibody. The antibodies in the kit may be labeled with one or more detectable markers, as described herein.

A kit may include one or more containers filled with one or more of the monoclonal antibodies of the disclosure. Additionally, the kit may include other reagents such as buffers and solutions needed to practice the disclosure are also included. Optionally associated with such container(s) may be a notice or printed instructions. As used herein, the phrase “packaging material” refers to one or more physical structures used to house the contents of the kit. The packaging material is constructed by well-known methods, preferably to provide a sterile, contaminant-free environment. As used herein, the term “package” refers to a solid matrix or material such as glass, plastic, paper, foil, and the like, capable of holding within fixed limits a polypeptide.

### Compositions Including Antibodies

In some embodiments, this disclosure describes a composition including at least one of the antibodies described herein.

In some embodiments, the composition may also include, for example, buffering agents to help to maintain the pH in an acceptable range or preservatives to retard microbial growth. A composition may also include, for example, carriers, excipients, stabilizers, chelators, salts, or antimicrobial agents. Acceptable carriers, excipients, stabilizers, chelators, salts, preservatives, buffering agents, or antimicrobial agents, include, but are not limited to, buffers such as phosphate, citrate, and other organic acids; antioxidants including ascorbic acid and methionine; preservatives, such as sodium azide, octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol; polypeptides; proteins, such as serum albumin, gelatin, or non-specific immunoglobulins; hydrophilic polymers such as olyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zinc (Zn)-protein complexes); and/or non-ionic surfactants such as TWEEN, PLURONICS, or polyethylene glycol (PEG).

In some embodiments, the composition is a pharmaceutical composition and includes the monoclonal antibody and a pharmaceutically acceptable carrier, diluent, or excipient. In the preparation of the pharmaceutical compositions comprising the antibodies described in the teachings herein, a variety of vehicles and excipients may be used, as will be apparent to the skilled artisan.

The pharmaceutical compositions will generally comprise a pharmaceutically acceptable carrier and a pharmacologically effective amount of an antibody, or mixture of antibodies.

The pharmaceutical composition may be formulated as a powder, a granule, a solution, a suspension, an aerosol, a solid, a pill, a tablet, a capsule, a gel, a topical cream, a suppository, a transdermal patch, and/or another formulation known in the art.

For the purposes described herein, pharmaceutically acceptable salts of an antibody are intended to include any art-recognized pharmaceutically acceptable salts including organic and inorganic acids and/or bases. Examples of salts include but are not limited to sodium, potassium, lithium, ammonium, calcium, as well as primary, secondary, and tertiary amines, esters of lower hydrocarbons, such as methyl, ethyl, and propyl. Other salts include but are not limited to organic acids, such as acetic acid, propionic acid, pyruvic acid, maleic acid, succinic acid, tartaric acid, citric acid, benzoic acid, cinnamic acid, salicylic acid, etc.

As used herein, “pharmaceutically acceptable carrier” comprises any standard pharmaceutically accepted carriers known to those of ordinary skill in the art in formulating pharmaceutical compositions. For example, the antibody may be prepared as a formulation in a pharmaceutically acceptable diluent, including for example, saline, phosphate buffer saline (PBS), aqueous ethanol, or solutions of glucose, mannitol, dextran, propylene glycol, oils (for example, vegetable oils, animal oils, synthetic oils, etc.), microcrystalline cellulose, carboxymethyl cellulose, hydroxylpropyl methyl cellulose, magnesium stearate, calcium phosphate, gelatin, polysorbate 80 or as a solid formulation in an appropriate excipient.

A pharmaceutical composition will often further comprise one or more buffers (for example, neutral buffered saline or phosphate buffered saline), carbohydrates (for example, glucose, sucrose or dextrans), mannitol, proteins, polypeptides or amino acids such as glycine, antioxidants (for example, ascorbic acid, sodium metabisulfite, butylated hydroxytoluene, butylated hydroxyanisole, etc.), bacteriostats, chelating agents such as EDTA or glutathione, adjuvants (for example, aluminum hydroxide), solutes that render the formulation isotonic, hypotonic or weakly hypertonic with the blood of a recipient, suspending agents, thickening agents and/or preservatives. Alternatively, compositions of the present disclosure may be formulated as a lyophilizate.

Any suitable carrier known to those of ordinary skill in the art may be employed in a composition including at least one of the antibodies describes herein. Antibody compositions may be formulated for any appropriate manner of administration, including for example, oral, nasal, mucosal, intravenous, intraperitoneal, intradermal, subcutaneous, and intramuscular administration.

### Administration and Treatment

In another aspect this disclosure describes a method that include administering an anti-SARS-CoV antibody, as described herein or a composition including an anti-SARS-CoV antibody, as described herein to a subject.

The subject may be any human or animal determined to benefit from the administration. For example, in some embodiments, the subject may be suspected of having SARS-CoV-1 or SARS-CoV-2 or may have been diagnosed with SARS-CoV-1 or SARS-CoV-2. In some embodiments, the subject may have been exposed to SARS-CoV-1 or SARS-CoV-2. In some embodiments, administration may result in preventing, slowing, and/or managing SARS or COVID-19 progression.

A composition including an anti-SARS-CoV antibody may be formulated in pharmaceutical preparations in a variety of forms adapted to the chosen route of administration. One of skill will understand that the composition will vary depending on mode of administration and dosage unit. For example, for parenteral administration, isotonic saline may be used. For topical administration a cream, including a carrier such as dimethylsulfoxide (DMSO), or other agents typically found in topical creams that do not block or inhibit activity of the peptide, may be used. Other suitable carriers include, but are not limited to alcohol, phosphate buffered saline, and other balanced salt solutions. The compounds of this disclosure may be administered in a variety of ways, including, but not limited to, intravenous, topical, oral, subcutaneous, intraperitoneal, and intramuscular delivery. In some embodiments, the compounds of the present disclosure may be formulated for controlled or sustained release. In some embodiments, a formulation for controlled or sustained release is suitable for subcutaneous implantation. In some embodiments, a formulation for controlled or sustained release includes a patch.

Administration may be as a single dose or in multiple doses. In some embodiments, the dose is an effective amount as determined by the standard methods, including, but not limited to, those described herein. Those skilled in the art of clinical trials will be able to optimize dosages of particular compounds through standard studies. Additionally, proper dosages of the compositions may be determined without undue experimentation using standard dose-response protocols. Administration includes, but is not limited to, any of the dosages and dosing schedules, dosing intervals, and/or dosing patterns described in the examples included herewith.

The composition including an antibody according to the present disclosure may be administered by any suitable means including, but not limited to, for example, oral, rectal, nasal, topical (including transdermal, aerosol, buccal and/or sublingual), vaginal, parenteral (including subcutaneous, intramuscular, and/or intravenous), intradermal, intravesical, intra-joint, intra-arteriole, intraventricular, intracranial, intraperitoneal, intranasal, by inhalation, or intralesional (for example, by injection into or around a tumor).

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that may be employed will be known to those of skill in the art. Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by the FDA. Such preparations may be pyrogen-free.

Many suitable formulations are known, including polymeric or protein microparticles encapsulating drug to be released, ointments, gels, or solutions which may be used topically or locally to administer drug, and even patches, which provide controlled release over a prolonged period of time. These may also take the form of implants. Such an implant may be implanted within the tumor.

The compounds of the present disclosure may also be provided in a lyophilized form. Such compositions may include a buffer, for example, bicarbonate, for reconstitution prior to administration, or the buffer may be included in the lyophilized composition for reconstitution with, for example, water. The lyophilized composition may further comprise a suitable vasoconstrictor, for example, epinephrine. The lyophilized composition may be provided in a syringe, optionally packaged in combination with the buffer for reconstitution, such that the reconstituted composition may be immediately administered to a patient.

As used herein “treating” or “treatment” may include therapeutic and/or prophylactic treatments. “Treating a disorder,” as used herein, is not intended to be an absolute term. Treatment may lead to an improved prognosis or a reduction in the frequency or severity of symptoms. A “therapeutically effective” concentration or amount as used herein is an amount that provides some improvement or benefit to the subject. Desirable effects of treatment include preventing occurrence or recurrence of disease, alleviation of symptoms, diminishment of any direct or indirect pathological consequences of the disease, decreasing the rate of disease progression, amelioration or palliation of the disease state, and remission or improved prognosis. Likewise, the term “preventing,” as used herein, is not intended as an absolute term. Instead, prevention refers to delay of onset, reduced frequency of symptoms, or reduced severity of symptoms associated with a disorder. Prevention therefore refers to a broad range of prophylactic measures that will be understood by those in the art. In some circumstances, the frequency and severity of symptoms is reduced to non-pathological levels. In some circumstances, the symptoms of an individual receiving the compositions of the disclosure are only 90%, 80%, 70%, 60%, 50%, 40%, 30%, 20%, 10%, 5%, or 1% as frequent or severe as symptoms experienced by an untreated individual with the disorder.

In some embodiments, the compounds of the present disclosure may be used for the presymptomatic treatment of individuals, with the administration of an anti-SARS-CoV antibody as described herein beginning after the determination or diagnosis of SARS or COVID-19, prior to the onset of symptoms. The diagnosis of SARS or COVID-19 may be made by any suitable method including, for example, antibody testing, PCR testing, etc.

Therapeutically effective concentrations and amounts may be determined for each application herein empirically by testing the compounds in known in vitro and in vivo systems, such as those described herein, dosages for humans or other animals may then be extrapolated therefrom.

It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions and methods.

Toxicity and therapeutic efficacy of the compositions may be determined by standard pharmaceutical procedures in cell cultures or experimental animals, for example, for determining the LD₅₀ (the dose lethal to 50% of the population) and the ED₅₀ (the dose therapeutically effective in 50% of the population). The dose ratio between toxic and therapeutic effects is the therapeutic index and it may be expressed as the ratio between LD₅₀ and ED₅₀. Compositions that exhibit high therapeutic indices may be preferred. The data obtained from cell culture assays and animal studies may be used in formulating a range of dosage for use in humans. The dosage of such compositions may preferably lie within a range of circulating concentrations that include the ED₅₀ with little or no toxicity. The dosage may vary within this range depending upon the dosage form employed and the route of administration utilized. The exact formulation, route of administration and dosage may be chosen by the individual physician in view of the patient's condition.

The dosage of such compositions may include similar doses as other antibody cocktails. Exemplary doses of antibody cocktails include, for example, antibody in a range of 25 mg/kg to 200 mg/kg including for example, 50 mg/kg of antibody or 150 mg/kg of antibody. If multiple antibodies are administered together, the dose may include 25 mg/kg to 75 mg/kg of each antibody, or, for example, 50 mg/kg of each antibody or 150 mg/kg of antibody. Exemplary doses for a human (having an average weight of 62 kg) may include a dose in a range of 1 g to 12 g of antibody, in a range of 1 g to 6 g of antibody, or in a range of 6 g to 12 g of antibody.

A composition as described herein may be administered at once or may be divided into a number of smaller doses to be administered at intervals of time. For example, compositions may be administered repeatedly, for example, at least 2, 3, 4, 5, 6, 7, 8, or more times, or may be administered by continuous infusion. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions and methods.

In some therapeutic embodiments, an “effective amount” of an agent is an amount that results in a reduction of at least one pathological parameter. Thus, for example, in some aspects of the present disclosure, an effective amount is an amount that is effective to achieve a reduction of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% compared to the expected reduction in the parameter in an individual not treated with the agent.

In some aspects of the methods of the present disclosure, a method further includes the administration of one or more additional therapeutic agents. One or more additional therapeutic agents may be administered before, after, and/or coincident to the administration of an antibody as described herein. An additional therapeutic agent may include, for example, convalescent serum, an anti-SARS-CoV monoclonal antibody, a small molecule pharmaceutical, a steroid, chemotherapy, radiation therapy, etc. Additional therapeutic agents may be administered separately or as part of a mixture or cocktail. In some aspects of the present disclosure, the administration of an antibody may allow for the effectiveness of a lower dosage of other therapeutic modalities when compared to the administration of the other therapeutic modalities alone, providing relief from the toxicity observed with the administration of higher doses of the other modalities.

In some aspects of the methods of the present disclosure, the administration of a composition as described herein and the at least one additional therapeutic agent demonstrate therapeutic synergy. In some aspects of the methods of the present disclosure, a measurement of response to treatment observed after administering both an antibody as described herein and the additional therapeutic agent is improved over the same measurement of response to treatment observed after administering either the antibody or the additional therapeutic agent alone.

The invention is defined in the claims. However, below there is provided a non-exhaustive listing of non-limiting exemplary aspects. Any one or more of the features of these aspects may be combined with any one or more features of another example, embodiment, or aspect described herein. Exemplary Embodiments of the present invention include, but are not limited to, the following.

1. An antigen binding molecule comprising an antibody variable domain with an amino acid sequence comprising:
at least 75%, at least 80%, at least 85%, at least 90%, at least 95%, at least 96%, at least 97%, at least 98%, or at least 99% sequence identity to QLQLVESGGGLVQPGGSLRLSCAASTLTLNSYAIGWFRQAPGKEREAVSCTSSSGDMTYY ANSVKGRFTISRDNAKNTVYLQMSRLKPEDTAVYYCAAVKLEYGYVCSHIPNEYDYWGQG TQVTVSS (SEQ ID NO: 1); or
at least 75%, at least 80%, at least 85%, at least 90%, at least 95%, at least 96%, at least 97%, at least 98%, or at least 99% sequence identity to

(SEQ ID NO: 2)
QLQLVESGGGLVQPGGSLRLSCAASTLTLNSYAIGWFRQAPGKEREAVS
CTSSSGDMTYYTNSVKGRFTISRDNAKNTVYLQMSRLKPDDTAVYYCAA
VLLEYGYVCSHPNEYDYWGQGTQVTVSS.

2. The antigen binding molecule of Embodiment 1, wherein the variable domain comprises an amino acid sequence comprising

(SEQ ID NO: 1)
QLQLVESGGGLVQPGGSLRLSCAASTLTLNSYAIGWFRQAPGKEREAVS
CTSSSGDMTYYANSVKGRFTISRDNAKNTVYLQMSRLKPEDTAVYYCAA
VKLEYGYVCSHPNEYDYWGQGTQVTVSS
or
(SEQ ID NO: 2)
QLQLVESGGGLVQPGGSLRLSCAASTLTLNSYAIGWFRQAPGKEREAVS
CTSSSGDMTYYTNSVKGRFTISRDNAKNTVYLQMSRLKPDDTAVYYCAA
VLLEYGYVCSHPNEYDYWGQGTQVTVSS.

3. An antigen binding molecule comprising an antibody variable domain comprising one or more complementary determining regions (CDRs) selected from:
a CDR1 sequence comprising an amino acid sequence which at least 75% sequence identity to SYAIG (SEQ ID NO: 3);
a CDR 2 sequence comprising an amino acid sequence with at least 75%, at least 80%, at least 85%, at least 90%, at least 95%, at least 96%, at least 97%, at least 98%, or at least 99% sequence identity to CTSSSGDMTYYANSVKG (SEQ ID NO: 4) or at least 75%, at least 80%, at least 85%, at least 90%, at least 95%, at least 96%, at least 97%, at least 98%, or at least 99% sequence identity to CTSSSGDMTYYTNSVKG (SEQ ID NO: 6); and
a CDR3 sequence comprising an amino acid sequence with at least 75%, at least 80%, at least 85%, at least 90%, at least 95%, at least 96%, at least 97%, at least 98%, or at least 99% sequence identity to VKLEYGYVCSHPNEYDY (SEQ ID NO: 5) or at least 75%, at least 80%, at least 85%, at least 90%, at least 95%, at least 96%, at least 97%, at least 98%, or at least 99% sequence identity to VLLEYGYVCSHPNEYDY (SEQ ID NO: 7).

4. An antigen binding molecule comprising an antibody variable domain comprising one or more complementary determining regions (CDRs) selected from:
a CDR1 sequence comprising an amino acid sequence which is identical to, or differs by one, two, or three amino acids from, SYAIG (SEQ ID NO: 3);
a CDR2 sequence comprising an amino acid sequence which is identical to, or differs by one, two, three, four, or five amino acids from, CTSSSGDMTYYANSVKG (SEQ ID NO: 4) or which is identical to, or differs by one, two, three, four, or five amino acids from CTSSSGDMTYYTNSVKG (SEQ ID NO: 6); and
a CDR3 sequence comprising an amino acid sequence which is identical to, or differs by one, two, three, four, or five amino acids from, VKLEYGYVCSHPNEYDY (SEQ ID NO: 5) or which is identical to, or differs by one, two, three, four, or five amino acids from VLLEYGYVCSHPNEYDY (SEQ ID NO: 7).

5. The antigen binding molecule of Embodiment 3 or 4, wherein the antigen binding molecule comprises an antibody variable domain comprising:
a CDR1 sequence comprising the amino acid sequence SYAIG (SEQ ID NO: 3); and
a CDR2 sequence comprising the amino acid sequence CTSSSGDMTYYANSVKG (SEQ ID NO: 4) or CTSSSGDMTYYTNSVKG (SEQ ID NO: 6); and
a CDR3 sequence comprising the amino acid sequence VKLEYGYVCSHPNEYDY (SEQ ID NO: 5) or VLLEYGYVCSHPNEYDY (SEQ ID NO: 7).

6. The antigen binding molecule of Embodiment 3 or 4, wherein the antigen binding molecule comprises an antibody variable domain comprising:
a CDR1 sequence comprising the amino acid sequence SYAIG (SEQ ID NO: 3); and
a CDR2 sequence comprising the amino acid sequence CTSSSGDMTYYANSVKG (SEQ ID NO: 4); and
a CDR3 sequence comprising the amino acid sequence VKLEYGYVCSHPNEYDY (SEQ ID NO: 5); or comprising:
a CDR1 sequence comprising the amino acid sequence SYAIG (SEQ ID NO: 3); and
a CDR2 sequence comprising the amino acid sequence CTSSSGDMTYYTNSVKG (SEQ ID NO: 6); and
a CDR3 sequence comprising the amino acid sequence VLLEYGYVCSHPNEYDY (SEQ ID NO: 7).

7. An antibody variable domain comprising an amino acid sequence with at least 75%, at least 80%, at least 85%, at least 90%, at least 95%, at least 96%, at least 97%, at least 98%, or at least 99% sequence identity to

(SEQ ID NO: 1)
QLQLVESGGGLVQPGGSLRLSCAASTLTLNSYAIGWFRQAPGKEREAVS
CTSSSGDMTYYANSVKGRFTISRDNAKNTVYLQMSRLKPEDTAVYYCAA
VKLEYGYVCSHPNEYDYWGQGTQVTVSS
or
(SEQ ID NO: 2)
QLQLVESGGGLVQPGGSLRLSCAASTLTLNSYAIGWFRQAPGKEREAVS
CTSSSGDMTYYTNSVKGRFTISRDNAKNTVYLQMSRLKPDDTAVYYCAA
VLLEYGYVCSHPNEYDYWGQGTQVTVSS.

8. The antibody variable domain of Embodiment 7, wherein the antibody variable domain comprises an amino acid sequence comprising

(SEQ ID NO: 1)
QLQLVESGGGLVQPGGSLRLSCAASTLTLNSYAIGWFRQAPGKEREAVS
CTSSSGDMTYYANSVKGRFTISRDNAKNTVYLQMSRLKPEDTAVYYCAA
VKLEYGYVCSHPNEYDYWGQGTQVTVSS
or
(SEQ ID NO: 2)
QLQLVESGGGLVQPGGSLRLSCAASTLTLNSYAIGWFRQAPGKEREAVS
CTSSSGDMTYYTNSVKGRFTISRDNAKNTVYLQMSRLKPDDTAVYYCAA
VLLEYGYVCSHPNEYDYWGQGTQVTVSS.

9. An antibody variable domain comprising one or more complementary determining regions (CDRs) selected from:
a CDR1 sequence comprising an amino acid sequence which at least 75% sequence identity to SYAIG (SEQ ID NO: 3);
a CDR 2 sequence comprising an amino acid sequence with at least 75%, at least 80%, at least 85%, at least 90%, at least 95%, at least 96%, at least 97%, at least 98%, or at least 99% sequence identity to CTSSSGDMTYYANSVKG (SEQ ID NO: 4) or at least 75%, at least 80%, at least 85%, at least 90%, at least 95%, at least 96%, at least 97%, at least 98%, or at least 99% sequence identity to CTSSSGDMTYYTNSVKG (SEQ ID NO: 6); and
a CDR3 sequence comprising an amino acid sequence with at least 75%, at least 80%, at least 85%, at least 90%, at least 95%, at least 96%, at least 97%, at least 98%, or at least 99% sequence identity to VKLEYGYVCSHPNEYDY (SEQ ID NO: 5) or at least 75%, at least 80%, at least 85%, at least 90%, at least 95%, at least 96%, at least 97%, at least 98%, or at least 99% sequence identity to VLLEYGYVCSHPNEYDY (SEQ ID NO: 7).

10. An antibody variable domain comprising one or more complementary determining regions (CDRs) selected from:
a CDR1 sequence comprising an amino acid sequence which is identical to, or differs by one, two, or three amino acids from, SYAIG (SEQ ID NO: 3);
a CDR2 sequence comprising an amino acid sequence which is identical to, or differs by one, two, three, four, or five amino acids from, CTSSSGDMTYYANSVKG (SEQ ID NO: 4) or which is identical to, or differs by one, two, three, four, or five amino acids from CTSSSGDMTYYTNSVKG (SEQ ID NO: 6); and
a CDR3 sequence comprising an amino acid sequence which is identical to, or differs by one, two, three, four, or five amino acids from, VKLEYGYVCSHPNEYDY (SEQ ID NO: 5) or which is identical to, or differs by one, two, three, four, or five amino acids from VLLEYGYVCSHPNEYDY (SEQ ID NO: 7).

11. The antibody variable domain of Embodiment 9 or 10, comprising:
a CDR1 sequence comprising the amino acid sequence SYAIG (SEQ ID NO: 3); and
a CDR2 sequence comprising the amino acid sequence CTSSSGDMTYYANSVKG (SEQ ID NO: 4) or CTSSSGDMTYYTNSVKG (SEQ ID NO: 6); and
a CDR3 sequence comprising the amino acid sequence VKLEYGYVCSHPNEYDY (SEQ ID NO: 5) or VLLEYGYVCSHPNEYDY (SEQ ID NO: 7).

12. The antibody variable domain of Embodiment 9 or 10 comprising:
a CDR1 sequence comprising the amino acid sequence SYAIG (SEQ ID NO: 3); and
a CDR2 sequence comprising the amino acid sequence CTSSSGDMTYYANSVKG (SEQ ID NO: 4); and
a CDR3 sequence comprising the amino acid sequence VKLEYGYVCSHPNEYDY (SEQ ID NO: 5);
or comprising:
a CDR1 sequence comprising the amino acid sequence SYAIG (SEQ ID NO: 3); and
a CDR2 sequence comprising the amino acid sequence CTSSSGDMTYYTNSVKG (SEQ ID NO: 6); and
a CDR3 sequence comprising the amino acid sequence VLLEYGYVCSHPNEYDY (SEQ ID NO: 7).

13. A camelid antibody single variable domain comprising an amino acid sequence with at least 75%, at least 80%, at least 85%, at least 90%, at least 95%, at least 96%, at least 97%, at least 98%, or at least 99% sequence identity to

(SEQ ID NO: 1)
QLQLVESGGGLVQPGGSLRLSCAASTLTLNSYAIGWFRQAPGKEREAVS
CTSSSGDMTYYANSVKGRFTISRDNAKNTVYLQMSRLKPEDTAVYYCAA
VKLEYGYVCSHPNEYDYWGQGTQVTVSS
or
(SEQ ID NO: 2)
QLQLVESGGGLVQPGGSLRLSCAASTLTLNSYAIGWFRQAPGKEREAVS
CTSSSGDMTYYTNSVKGRFTISRDNAKNTVYLQMSRLKPDDTAVYYCAA
VLLEYGYVCSHPNEYDYWGQGTQVTVSS.

14. The camelid antibody single variable domain of Embodiment 13, wherein the camelid antibody single variable domain comprises an amino acid sequence comprising

(SEQ ID NO: 1)
QLQLVESGGGLVQPGGSLRLSCAASTLTLNSYAIGWFRQAPGKEREAVS
CTSSSGDMTYYANSVKGRFTISRDNAKNTVYLQMSRLKPEDTAVYYCAA
VKLEYGYVCSHPNEYDYWGQGTQVTVSS
or
(SEQ ID NO: 2)
QLQLVESGGGLVQPGGSLRLSCAASTLTLNSYAIGWFRQAPGKEREAVS
CTSSSGDMTYYTNSVKGRFTISRDNAKNTVYLQMSRLKPDDTAVYYCAA
VLLEYGYVCSHPNEYDYWGQGTQVTVSS.

15. A camelid antibody single variable domain comprising one or more complementary determining regions (CDRs) selected from:
a CDR1 sequence comprising an amino acid sequence which at least 75% sequence identity to SYAIG (SEQ ID NO: 3);
a CDR 2 sequence comprising an amino acid sequence with at least 75%, at least 80%, at least 85%, at least 90%, at least 95%, at least 96%, at least 97%, at least 98%, or at least 99% sequence identity to CTSSSGDMTYYANSVKG (SEQ ID NO: 4) or at least 75%, at least 80%, at least 85%, at least 90%, at least 95%, at least 96%, at least 97%, at least 98%, or at least 99% sequence identity to CTSSSGDMTYYTNSVKG (SEQ ID NO: 6); and
a CDR3 sequence comprising an amino acid sequence with at least 75%, at least 80%, at least 85%, at least 90%, at least 95%, at least 96%, at least 97%, at least 98%, or at least 99% sequence identity to VKLEYGYVCSHPNEYDY (SEQ ID NO: 5) or at least 75%, at least 80%, at least 85%, at least 90%, at least 95%, at least 96%, at least 97%, at least 98%, or at least 99% sequence identity to VLLEYGYVCSHPNEYDY (SEQ ID NO: 7).

16. A camelid antibody single variable domain comprising one or more complementary determining regions (CDRs) selected from:
a CDR1 sequence comprising an amino acid sequence which is identical to, or differs by one, two, or three amino acids from, SYAIG (SEQ ID NO: 3);
a CDR2 sequence comprising an amino acid sequence which is identical to, or differs by one, two, three, four, or five amino acids from, CTSSSGDMTYYANSVKG (SEQ ID NO: 4) or which is identical to, or differs by one, two, three, four, or five amino acids from CTSSSGDMTYYTNSVKG (SEQ ID NO: 6); and
a CDR3 sequence comprising an amino acid sequence which is identical to, or differs by one, two, three, four, or five amino acids from, VKLEYGYVCSHPNEYDY (SEQ ID NO: 5) or which is identical to, or differs by one, two, three, four, or five amino acids from VLLEYGYVCSHPNEYDY (SEQ ID NO: 7).

17. The camelid antibody single variable domain of Embodiment 15 or 16, comprising:
a CDR1 sequence comprising the amino acid sequence SYAIG (SEQ ID NO: 3); and
a CDR2 sequence comprising the amino acid sequence CTSSSGDMTYYANSVKG (SEQ ID NO: 4) or CTSSSGDMTYYTNSVKG (SEQ ID NO: 6); and
a CDR3 sequence comprising the amino acid sequence VKLEYGYVCSHPNEYDY (SEQ ID NO: 5) or VLLEYGYVCSHPNEYDY (SEQ ID NO: 7).

18. The camelid antibody single variable domain of Embodiment 15 or 16 comprising:
a CDR1 sequence comprising the amino acid sequence SYAIG (SEQ ID NO: 3); and
a CDR2 sequence comprising the amino acid sequence CTSSSGDMTYYANSVKG (SEQ ID NO: 4); and
a CDR3 sequence comprising the amino acid sequence VKLEYGYVCSHPNEYDY (SEQ ID NO: 5);
or comprising:
a CDR1 sequence comprising the amino acid sequence SYAIG (SEQ ID NO: 3); and
a CDR2 sequence comprising the amino acid sequence CTSSSGDMTYYTNSVKG (SEQ ID NO: 6); and
a CDR3 sequence comprising the amino acid sequence VLLEYGYVCSHPNEYDY (SEQ ID NO: 7).

19. A camelid heavy chain IgG (hcIgG) antibody comprising a camelid antibody single variable domain of any one of Embodiments 13 to 18.

20. The camelid heavy chain hcIgG antibody of Embodiment 19, comprising a llama heavy chain IgG (hcIgG) antibody.

21. The antibody binding molecule, antibody variable domain, camelid antibody single variable domain, or hcIgG antibody camelid antibody of any one of the preceding Embodiments, wherein the antibody binding molecule, antibody variable domain, camelid antibody single variable domain, or hcIgG antibody specifically binds the SARS-CoV-2 spike protein 51.

22. The antibody binding molecule, antibody variable domain, camelid antibody single variable domain, or hcIgG antibody camelid antibody of any one of the preceding Embodiments, wherein the antibody binding molecule, antibody variable domain, camelid antibody single variable domain, or hcIgG antibody specifically binds to the receptor binding (RBD) domain (amino acids 319 to 541) of the SARS-CoV-2 spike protein 51.

23. The antibody binding molecule, antibody variable domain, camelid antibody single variable domain, or hcIgG antibody of any one of the preceding Embodiments, wherein the antibody binding molecule, antibody variable domain, camelid antibody single variable domain, or hcIgG antibody inhibits binding of SARS-CoV-1 and/or SARS-CoV-2 to ACE-2.

24. The antibody binding molecule, antibody variable domain, camelid antibody single variable domain, or hcIgG antibody of Embodiment 23, wherein the binding of SARS-CoV-1 or SARS-CoV-2 or both SARS-CoV-1 and SARS-CoV-2 to ACE-2 is decreased by at least 10 percent (%), at least 15%, at least 20%, at least 25%, at least 30%, at least 40%, at least 50%, at least 60%, at least 70%, at least 80%, at least 90%, at least 95%, or at least 98%.

25. The antibody binding molecule, antibody variable domain, camelid antibody single variable domain, or hcIgG antibody of any one of the preceding Embodiments, wherein the antibody binding molecule, antibody variable domain, camelid antibody single variable domain, or hcIgG antibody is humanized.

26. The antibody binding molecule, antibody variable domain, camelid antibody single variable domain, or hcIgG antibody of any one of the preceding Embodiments further comprising a human immunoglobulin constant region.

27. The antibody binding molecule, antibody variable domain, camelid antibody single variable domain, or hcIgG antibody of any one of the preceding Embodiments conjugated to a small molecule or protein.

28. The antibody binding molecule, antibody variable domain, camelid antibody single variable domain, or hcIgG antibody of any one of the preceding Embodiments conjugated to a detectable marker.

29. A bispecific or multivalent antibody comprising the antibody binding molecule, antibody variable domain, camelid antibody single variable domain, or hcIgG antibody of any one of the preceding Embodiments.

30. A composition comprising the antibody binding molecule, antibody variable domain, camelid antibody single variable domain, or hcIgG antibody of any one of the preceding Embodiments.

31. The composition of Embodiment 30, the composition further comprising at least one additional anti-SARS-CoV antibody.

32. The composition of Embodiment 30 or Embodiment 31, the composition further comprising a pharmaceutically acceptable carrier.

33. A method comprising administering the antibody binding molecule, antibody variable domain, camelid antibody single variable domain, or hcIgG antibody of any one of Embodiments 1 to 29 or the composition of any one of Embodiments 30 to 32 to a subject.

34. The method of Embodiment 33, wherein the subject is suspected of having SARS-CoV-1 or SARS-CoV-2 or has been diagnosed with SARS-CoV-1 or SARS-CoV-2.

35. The method of Embodiment 33 or 34, wherein the subject has been exposed to SARS-CoV-1 or SARS-CoV-2.

36. The method of any one of Embodiments 33 to 35, wherein the subject is a human.

37. The method of any one of Embodiments 33 to 36, comprises administering multiple doses of the antibody binding molecule, antibody variable domain, camelid antibody single variable domain, or hcIgG antibody to a subject.

38. A method comprising using the antibody binding molecule, antibody variable domain, camelid antibody single variable domain antibody, or hcIgG antibody of any one of Embodiments 1 to 29 or the composition of any one of Embodiments 30 to 32 to diagnose a subject with SARS-CoV-1 or SARS-CoV-2.

39. A nucleotide sequence encoding the antibody binding molecule, antibody variable domain, camelid antibody single variable domain, or hcIgG antibody of any one of Embodiments 1 to 29.

40. An expression vector comprising the nucleotide sequence of Embodiment 39.

41. A host cell transformed with the nucleotide sequence of Embodiment 39 or the expression vector of Embodiment 40.

42. A method for producing an antibody binding molecule, antibody variable domain, camelid antibody single variable domain antibody, or hcIgG antibody, the method comprising culturing the host cell of Embodiment 41 under conditions that allow the host cell to translate the nucleotide sequence encoding the antibody binding molecule, camelid antibody single variable domain, or hcIgG antibody, thereby producing the antibody binding molecule, antibody variable domain, camelid antibody single variable domain antibody, or hcIgG antibody.

43. The method of Embodiment 42, further comprising harvesting, purifying and/or isolating said antibody binding molecule, antibody variable domain, camelid antibody single variable domain antibody, or hcIgG antibody.

The present invention is illustrated by the following examples. It is to be understood that the particular examples, materials, amounts, and procedures are to be interpreted broadly in accordance with the scope and spirit of the invention as set forth herein.

### EXAMPLES

All reagents, starting materials, and solvents used in the following examples were purchased from commercial suppliers (such as Sigma Aldrich, St. Louis, MO) and were used without further purification unless otherwise indicated.

### Example 1

This Example describes the isolation and characterization of the 70009-1 and 70009-2 single-domain camelid antibodies specific to the SARS-CoV2/S1 RBD.

### Materials and Methods

### Llama Immunization Protocol

To obtain single-domain camelid antibodies specific to the SARS-CoV2/S1 RBD protein, a llama was immunized subcutaneously with the SARS-CoV2/S1 RBD Protein (UniProtKB/SwissProt Accession No. PODTC2), supplied at 0.4 mg/mL with 0.08 mg/mL CpG (ODN 1826) as follows:
Day 1—First injection with 0.5 mg in 1.2 mL and 3.6 ml complete Freund's adjuvant
Day 14—Boost with 0.5 mg in 1.2 mL and 3.6 ml incomplete Freund's adjuvant
Day 28—Boost with 0.5 mg in 1.2 mL and 3.6 ml incomplete Freund's adjuvant
Day 49—Boost with 0.5 mg in 1.2 mL and 3.6 ml incomplete Freund's adjuvant
Day 79—Bost with 0.5 mg in 1.2 mL and 3.6 ml incomplete Freund's adjuvant

### B-Cell Isolation

Antigen-specific B cells were isolated from 30 ml of heparinized llama blood using MagCellect Magnet protocols. For the preparation of a B cell library, a suspension of 5×10⁶ cells per 1 ml is pelleted and resuspended in 100 μl lysis buffer (Qiagen) and kept at −80° C. until ready for molecular cloning.

### Cloning and Transient Expression

Total RNA was isolated from antigen-specific B cells, reverse-transcribed to cDNA, and used as template in PCR to amplify llama VHH transcripts. VHH-specific PCR amplicons were cloned into an expression vector to promote soluble expression in HEK293 cells. Individual clones, corresponding to transfection medias with specificity to SARS COV-2 RBD in direct ELISA, were selected for sequence analysis and further characterized.

Two clones, 70009-1 and 70009-2, were selected for further characterization.

### Antibody Characterization

Construction of hACE-2 HEK eGFP and hCD26 HEK eGFP Transfectants

HEK-293 wild-type cells were transfected with expression plasmids containing a hACE-2 (amino acids 1-708; Accession No. Q9BYF1) cDNA insert and eGFP cDNA insert (amino acids 1-239; Accession No. U57607) downstream of a CMV promoter or a hCD26 (amino acids 1-766; Accession No. Q53TN1) cDNA insert and eGFP cDNA insert (amino acids 1-239; Accession No. U57607) downstream of a CMV promoter. Stable clones expressing hACE-2/eGFP or hCD26/eGFP were used in all assays.

The resulting GFP-labeled HEK transfectant cells stably over-expressing human ACE-2 (“hACE-2 HEK/eGFP Tfx”) and GFP-labeled HEK transfectant cells stably over-expressing human CD26 (“hCD26 HEK/eGFP Tfx”) were grown in IMDM complete selection media (5% FBS, 1× Pen/Strep, and 1 μg/mL puromycin).

hACE-2 HEK/eGFP Tfx were screened periodically with anti-hACE-2 antibody (MAB9332, Bio-Techne, Minneapolis, MN) to ensure continued high expression of hACE-2.

### SARS-CoV-2 Antibody Blocking Assays

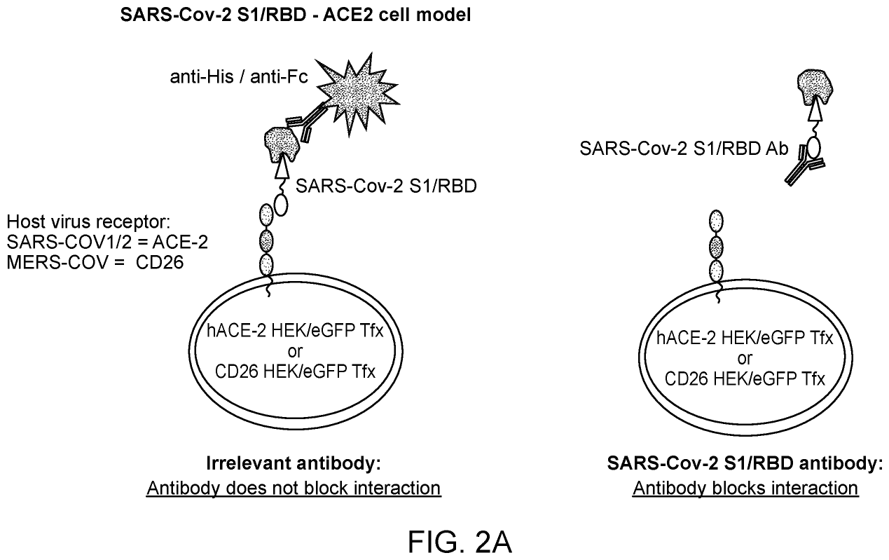

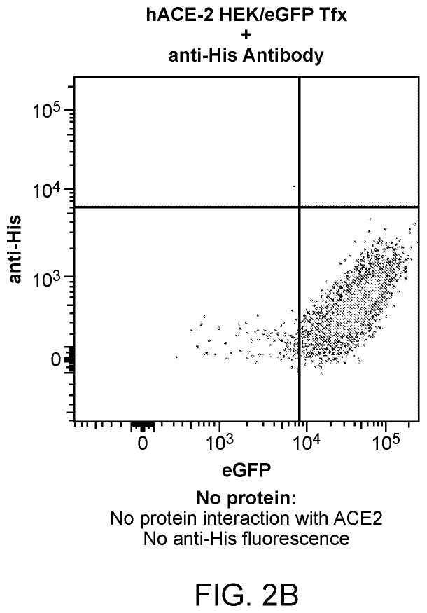

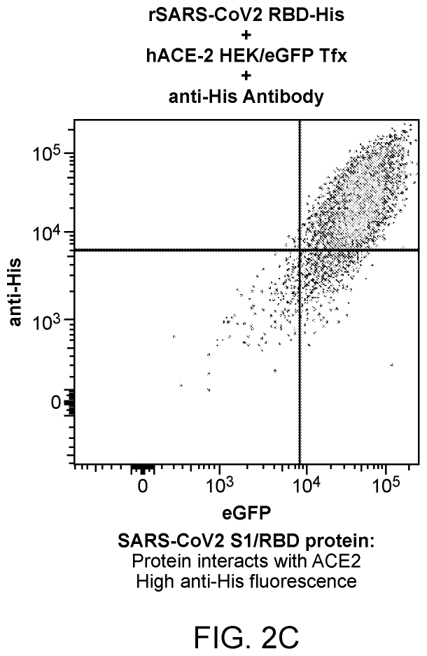

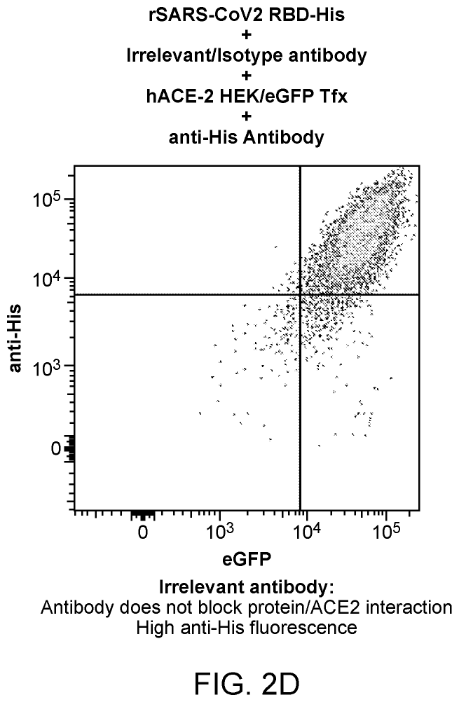

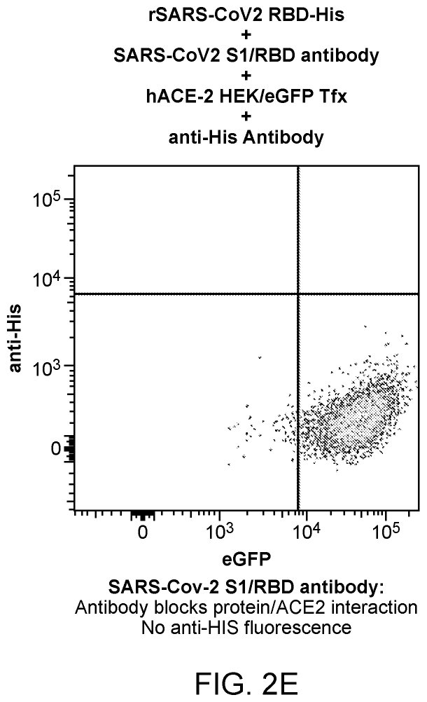

A schematic description of the SARS-CoV-2 antibody blocking assay is shown in FIGS. 2A-2E, using a hACE-2 HEK/eGFP Tfx and rSARS-CoV-2 S1 RBD protein for illustrative purposes.

The blocking assays described herein used hACE-2 HEK/eGFP Tfx or hCD26 HEK/eGFP Tfx for the cell model; rSARS-CoV-2 wild type or mutation variants, rSARS-CoV-1, or MERS proteins as the protein; and a Recombinant LLAMABODY VHH antibody (70009-1). Antibody tested was added at a final concentration of 25 μg/mL. Proteins were added at a final concentration ranging from 50 ng/mL to 1 μg/mL.

Viral proteins/antibodies were co-incubated to form a complex and then added to hACE-2 HEK/eGFP Tfx or hCD26 HEK/eGFP Tfx.

Anti-His APC or anti-Fc-APC was added to the antibody/protein/Tfx samples for detection. The samples were then washed, a live/dead stain was added to exclude dead cells, and analysis was carried out on a BD LSRFortessa™

Negative controls included hACE-2 HEK/eGFP Tfx or hCD26 HEK/eGFP Tfx alone; anti-His APC+(hACE-2 HEK/eGFP Tfx or hCD26 HEK/eGFP Tfx); and isotype controls (Llama IgG) +protein+(hACE-2 HEK/eGFP Tfx or hCD26 HEK/eGFP Tfx)+anti-His APC. rSARS-CoV-2 S1, rSARS-CoV-2 S1 RBD, rSARS-CoV-2 active trimer, rSARS-CoV-1 S1, or rSARS-CoV-1 S1 RBD+(hACE-2 HEK/eGFP Tfx or hCD26 HEK/eGFP Tfx)+anti-His APC served as a protein binding control for rSARS-CoV-1 and rSARS-CoV-2.

rMERS S1+(hACE-2 HEK/eGFP Tfx or hCD26 HEK/eGFP Tfx)+anti-Fc APC, or rMERS S1 RBD+(hACE-2 HEK/eGFP Tfx or hCD26 HEK/eGFP Tfx)+anti-His APC served as the protein binding controls for rMERS.

Anti-viral antibodies+viral proteins+(hACE-2 HEK/eGFP Tfx or hCD26 HEK/eGFP Tfx) +(anti-His APC or anti-Fc APC) were used to assess viral antibody blocking of hACE-2 vs. SARS-CoV-2 or SARS-CoV-1 proteins, or antibody blocking of hCD26 vs. MERS proteins.

### Results

### Blockade of SARS-Cov2 RBD, S1, and Variants Protein Binding to ACE2 Receptor Expressing Cells Inflow Cytometry Assay

FIG. 2 is a schematic of a SARS-CoV-2 viral antibody blocking cell model, using a hACE-2 HEK/eGFP Tfx and rSARS-CoV-2 S₁ RBD protein for illustrative purposes. FIG. 2A shows a schematic of SARS-CoV-2 viral antibody blocking cell model. An antibody and a recombinant SARS-CoV-2 S₁ RBD-His protein are exposed to one another, allowing a complex to form between the antibody (for example, an anti-SARS-CoV-2 antibody) and the SARS-CoV-2 S₁ RBD-His protein if the antibody can bind to the SARS-CoV-2 S₁ RBD-His protein. This mixture (including, if one formed, an antibody-SARS-CoV-2 S₁ RBD-His protein complex) is added to a GFP-labeled HEK transfectant cell stably over-expressing human ACE-2 (hACE-2 HEK/eGFP Tfx). Binding between the SARS-CoV-2 S₁ RBD-His protein and the human ACE-2 is detected using an anti-His antibody. Exemplary results are shown in FIG. 2B-FIG. 2E. In the absence of a SARS-CoV-2 protein, no anti-His fluorescence is detected by flow cytometry (FIG. 2B); however, when a SARS-CoV-2 S₁ RBD protein is added to hACE-2 HEK/eGFP cells, a high level of anti-His fluorescence is detected (FIG. 2C). In the presence of irreverent or isotype control antibodies SARS-CoV-2 S₁/RBD proteins bind to ACE-2, and fluorescence remains high (FIG. 2D). In contrast, when anti-SARS-CoV-2 S₁ RBD antibodies are added, the antibodies form a complex with SARS-CoV-2 S₁/RBD proteins, preventing the viral proteins from binding ACE-2, resulting in decreased anti-His fluorescence (FIG. 2E).

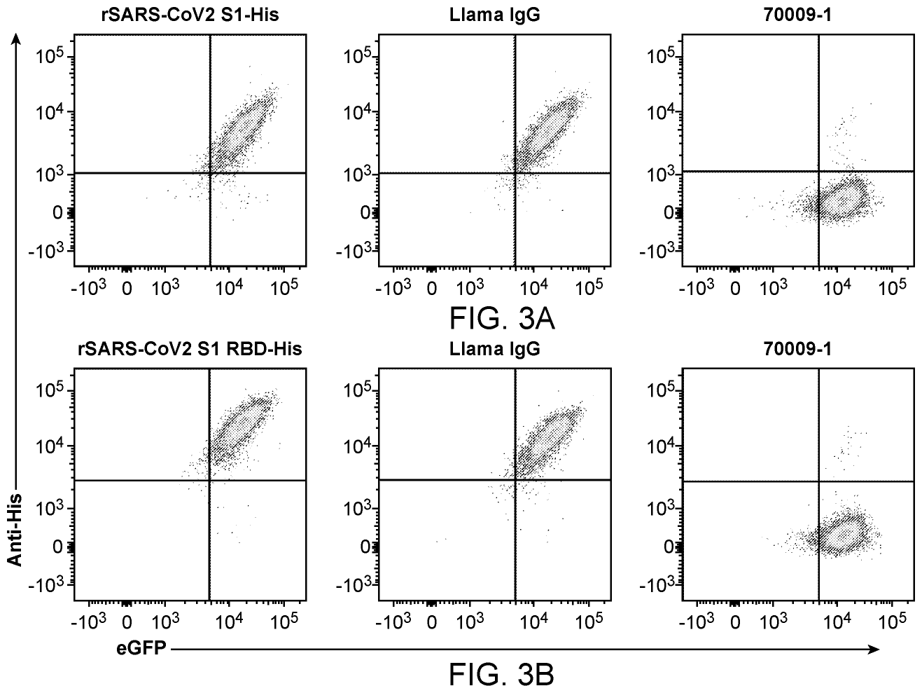

SARS-CoV-2 S1 (100 ng/mL) (FIG. 3A) or SARS-CoV-2 S1 RBD (50 ng/mL) (FIG. 3B) His-tagged protein was incubated with hACE-2/eGFP cells in the absence of antibody (left panels), or in the presence of 25 μg/mL llama IgG control antibody, Recombinant LLAMABODY VHH antibody (70009-1). Protein binding was detected with fluorescently labeled anti-His monoclonal antibody and analyzed by flow cytometry. When a SARS-CoV-2 S1 or RBD protein is added to hACE-2 HEK/eGFP cells, a high level of anti-His fluorescence is detected (left panels). In the presence of irrelevant or isotype control antibodies SARS-CoV-2 S1/RBD proteins bind to ACE-2, and fluorescence remains high (llama IgG). In contrast, when anti-SARS-CoV-2 S1 RBD antibody is added, the antibodies form a complex with SARS-CoV-2 S1/RBD proteins, preventing the viral proteins from binding ACE-2, resulting in decreased anti-His fluorescence (70009-1).

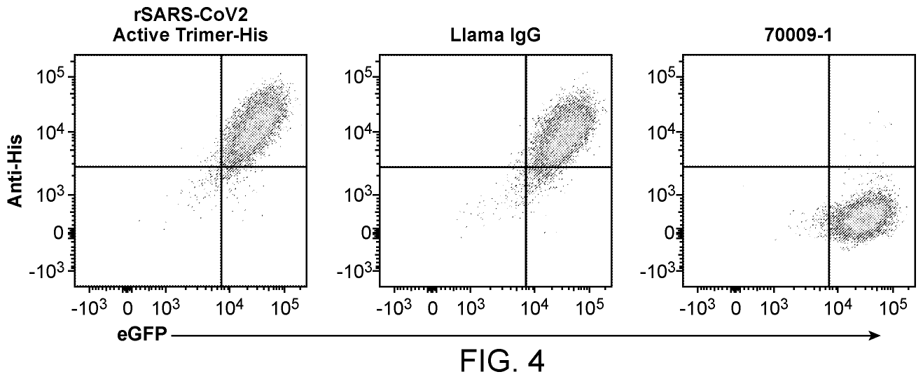

As shown in FIG. 4, SARS-CoV-2 Active Trimer (500 ng/mL) His-tagged protein was incubated with hACE-2/eGFP cells in the absence of antibody (left panels), or in the presence of 25 μg/mL llama IgG control antibody or Recombinant LLAMABODY VHH antibody (70009-1). Protein binding was detected with fluorescently labeled anti-His monoclonal antibody and analyzed by flow cytometry. Anti-SARS-CoV-2 S1 RBD antibody 70009-1 blocks binding of SARS-CoV-2 Active Trimer to hACE-2/eGFP cells.

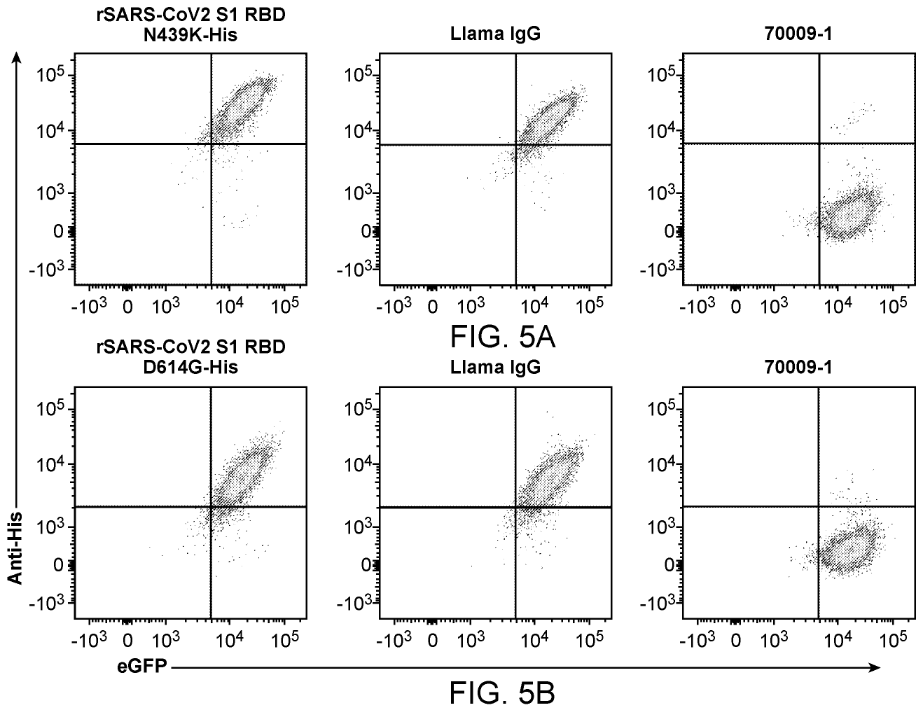

SARS-CoV-2 N439K variant (50 ng/mL) (FIG. 5A) or SARS-CoV-2 D614G variant (100 ng/mL) (FIG. 5B) His-tagged protein was incubated with hACE-2/eGFP cells in the absence of antibody (left panels), or in the presence of 25 μg/mL llama IgG control antibody or Recombinant LLAMABODY VHH antibody (70009-1). Protein binding was detected with fluorescently labeled anti-His monoclonal antibody and analyzed by flow cytometry. Anti-SARS-CoV-2 S1 RBD antibody 70009-1 blocks binding of SARS-CoV-2 N439K and D614G variants to hACE-2/eGFP cells.

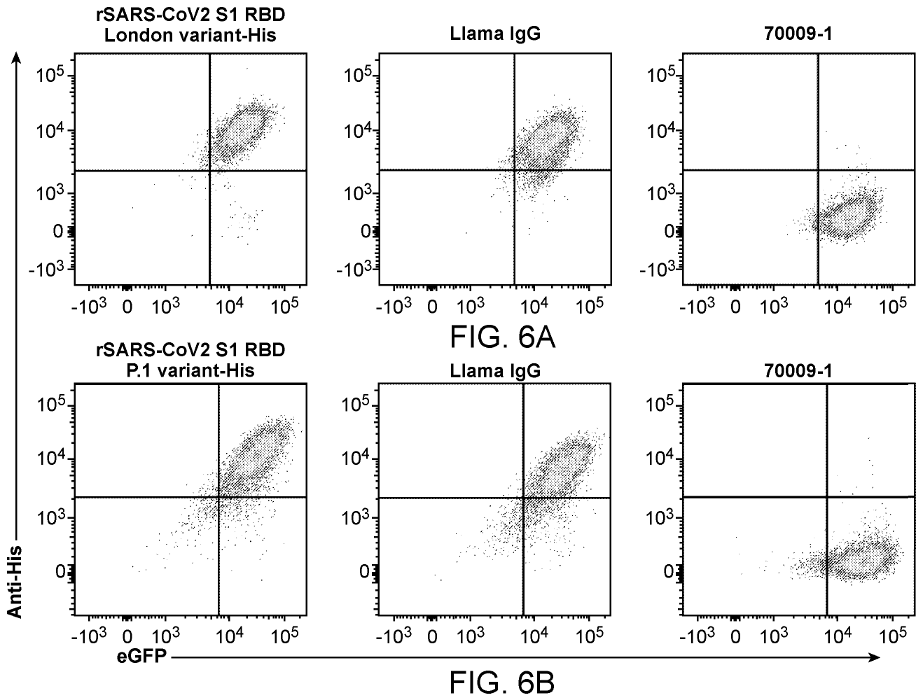

SARS-CoV-2 London variant (500 ng/mL) (FIG. 6A) or SARS-CoV-2 P.1 variant (50 ng/mL) (FIG. 6B) His-tagged protein was incubated with hACE-2/eGFP cells in the absence of antibody (left panels), or in the presence of 25 μg/mL llama IgG control antibody or Recombinant LLAMABODY VHH antibody (70009-1). Protein binding was detected with fluorescently labeled anti-His monoclonal antibody and analyzed by flow cytometry. Anti-SARS-CoV-2 S1 RBD antibody 70009-1 blocks binding of SARS-CoV-2 London and P.1 variants to hACE-2/eGFP cells.

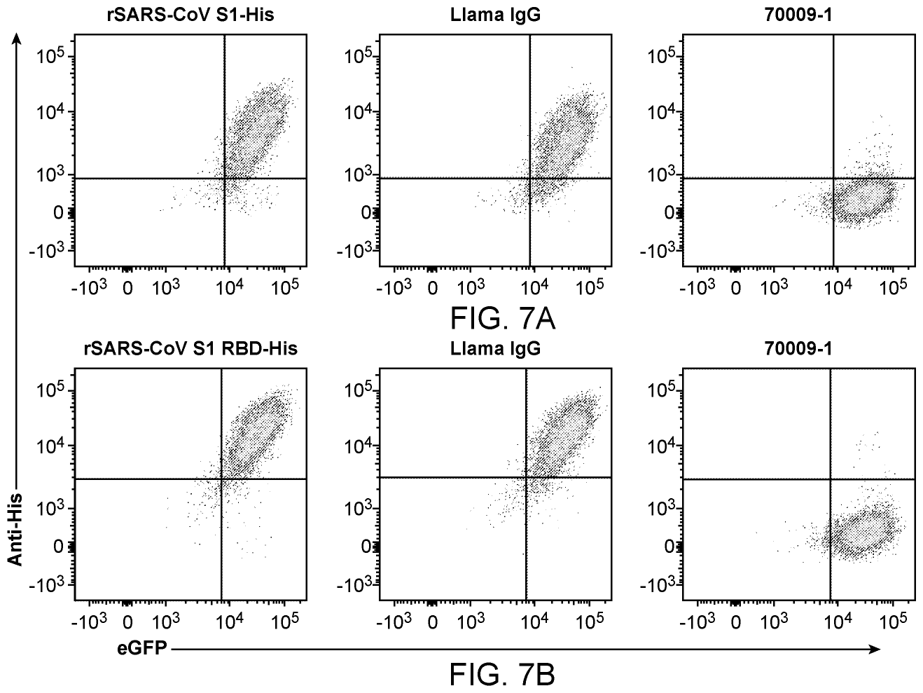

SARS-CoV S1 (100 ng/mL) (FIG. 7A) or SARS-CoV S₁ RBD (50 ng/mL) (FIG. 7B) His-tagged protein was incubated with hACE-2/eGFP cells in the absence of antibody (left panels), or in the presence of 25 μg/mL llama IgG control antibody or Recombinant LLAMABODY VHH antibody (70009-1) to assess whether these antibodies could cross react with SARS-Cov. Protein binding was detected with fluorescently labeled anti-His monoclonal antibody and analyzed by flow cytometry. Anti-SARS-CoV-2 S1 RBD antibody 70009-1 blocks binding of SARS-CoV S1 and S1 RBD to hACE-2/eGFP cells.

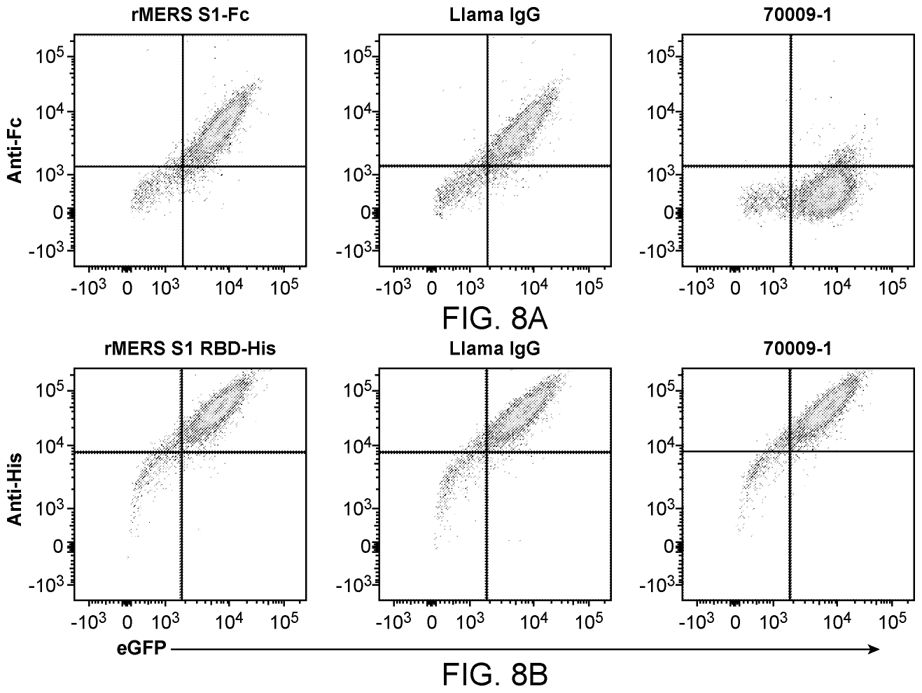

MERS S1 (1 μg/mL) (FIG. 8A) or MERS S1 RBD (1 μg/mL) (FIG. 8B) Fc-tagged protein was incubated with hCD26/eGFP cells in the absence of antibody (left panels), or in the presence of 25 μg/mL llama IgG control antibody or Recombinant LLAMABODY VHH antibody (70009-1) to assess whether these antibodies could cross react with MERS. Protein binding was detected with fluorescently labeled anti-Fc monoclonal antibody and analyzed by flow cytometry. Anti-SARS-CoV-2 S1 RBD antibody 70009-1 blocks binding of MERS S1 but not S1 RBD to hCD26/eGFP cells.

### Summary of Llama SARS-CoV2 Blocking for the 70009-1 Antibody.

Antibody blocking of a recombinant monomeric SARS-CoV-2 S1-ACE-2 interaction and a recombinant monomeric SARS-CoV-2 S1 RBD-ACE-2 interaction is shown below in Table 1. Percent Blocking was calculated by subtracting the percent of anti-His positive cells in the presence of monoclonal antibody from the percent of anti-His positive cells incubated with either S1 or S1 RBD. 100% blocking would indicate an antibody that completely prevented the binding of SARS-CoV-2 S1 or SARS-CoV-2 S1 RBD to ACE-2.

TABLE 1
Antibody blocking of a recombinant monomeric SARS-
CoV-2 S₁-ACE-2 interaction and a recombinant monomeric
SARS-CoV-2 S₁ RBD-ACE-2 interaction

| Target | Fusion | SARS-CoV2 S1 blocking | SARS-CoV2 RBD blocking |
|---|---|---|---|
| RBD | 70009-1 | 97.0% | 98.5% |

Antibody blocking of a recombinant homotrimeric SARS-CoV-2 Spike protein-ACE-2 interaction is shown below in Table 2. Percent Blocking was calculated by subtracting the percent of anti-His positive cells in the presence of monoclonal antibody from the percent of anti-His positive cells incubated with homotrimeric spike protein alone. 100% blocking would indicate an antibody that completely prevented the binding of SARS-CoV-2 spike protein to ACE-2.

TABLE 2
Antibody blocking of a recombinant homotrimeric
SARS-CoV-2 Spike protein-ACE-2 interaction

| Target | Fusion | SARS-CoV2 active trimer blocking |
|---|---|---|
| RBD | 70009-1 | 96.2% |

Table 3 below is a summary of anti-SARS-CoV-2 antibody blocking of recombinant SARS-CoV-2 S1 RBD N439K, D614G, London variant, and P1 variants.

TABLE 3
Summary of anti-SARS-CoV-2 antibody blocking of recombinant SARS-
CoV-2 S₁ RBD N439K, D614G, London variant, and P1 variants

| | | SARS-CoV2 RBD | SARS-CoV2 RBD | SARS-CoV2 RBD | SARS-CoV2 RBD |
|---|---|---|---|---|---|
| Target | Fusion | N439K blocking | D614G blocking | London* blocking | P.1 blocking |
| RBD | 70009-1 | 97.5% | 94.1% | 98.0% | Not done |

Antibody blocking of a recombinant SARS-CoV-1 S1-ACE-2 interaction and a recombinant SARS-CoV-1 S1 RBD-ACE-2 interaction is shown in Table 4. Percent blocking was calculated by subtracting the percent of anti-His positive cells in the presence of monoclonal antibody from the percent of anti-His positive cells incubated with either S1 or S1 RBD. 100% blocking would indicate an antibody that completely prevented the binding SARS-CoV-1 S1 or SARS-CoV-1 S1 RBD to ACE-2.

TABLE 4
Antibody blocking of a recombinant SARS-CoV-1 S₁-ACE-2 interaction
and a recombinant SARS-CoV-1 S₁ RBD-ACE-2 interaction

| Target | Fusion | SARS-CoV S1 blocking | SARS-CoV RBD blocking |
|---|---|---|---|
| RBD | 70009-1 | 92.5% | 96.3% |

Antibody blocking of a recombinant MERS S1-CD26 interaction and a recombinant MERS S1 RBD-CD26 interaction is shown below in Table 5. Percent blocking was calculated by subtracting the percent of anti-His positive cells in the presence of monoclonal antibody from the percent of anti-His positive cells incubated with either S1 or S1 RBD. 100% blocking would indicate an antibody that completely prevented the binding MERS S1 or MERS S1 RBD to CD26.

TABLE 5
Antibody blocking of a recombinant MERS S₁-CD26 interaction
and a recombinant MERS S₁ RBD-CD26 interaction

| Target | Fusion | MERS S1 blocking | MERS RBD blocking |
|---|---|---|---|
| RBD | 70009-1 | 87.2% | 0 |

Table 6 below is a summary of anti-SARS-CoV-2 antibody blocking of recombinant SARS-CoV-2 S1, SARS-CoV-2 S1 RBD and variants, trimeric SARS-CoV-2 spike protein, SARS-CoV-1 S1, SARS-CoV-1 S1 RBD, MERS S1, and MERS S1 RBD.

TABLE 6
Summary of anti-SARS-CoV-2 antibody blocking of recombinant
SARS-CoV-2 S₁, SARS-CoV-2 S₁ RBD and variants, trimeric
SARS-CoV-2 spike protein, SARS-CoV-1 S₁, SARS-CoV-1 S₁ RBD,
MERS S1, and MERS S₁ RBD

| Target | RBD |
|---|---|
| Fusion | 70009-1 |
| SARS-CoV2 S1 blocking | 97.0% |
| SARS-CoV2 RBD blocking | 98.5% |
| SARS-CoV2 active trimer blocking | 96.2% |
| SARS-CoV2 RBD N439K blocking | 97.5% |
| SARS-CoV2 RBD D614G blocking | 94.1% |
| SARS-CoV2 RBD London variant | 98.0% |
| SARS-CoV2 RBD P.1 blocking | Not done |
| SARS-CoV S1 blocking | 92.5% |
| SARS-CoV RBD blocking | 96.3% |
| MERS S1 blocking | 87.2% |
| MERS RBD blocking | 0 |

### Example 2

The 70009-1 and 70009-2 VHH clones described in Example 1 were sequenced. The amino acid sequences of the 70009-1 and 70009-2 VHH clones are given below.

70009-1
(SEQ ID NO: 1)
QLQLVESGGGLVQPGGSLRLSCAASTLTLNSYAIGWFRQAPGKEREAVS
CTSSSGDMTYYANSVKGRFTISRDNAKNTVYLQMSRLKPEDTAVYYCAA
VKLEYGYVCSHPNEYDYWGQGTQVTVSS
70009-2
(SEQ ID NO: 2)
QLQLVESGGGLVQPGGSLRLSCAASTLTLNSYAIGWFRQAPGKEREAVS
CTSSSGDMTYYTNSVKGRFTISRDNAKNTVYLQMSRLKPDDTAVYYCAA
VLLEYGYVCSHPNEYDYWGQGTQVTVSS

The complementarity determining regions are as follows:

70009-1
HC CDR1
(SEQ ID NO: 3)
SYAIG
HC CDR2
(SEQ ID NO: 4)
CTSSSGDMTYYANSVKG
HC CDR3
(SEQ ID NO: 5)
VKLEYGYVCSHPNEYDY
70009-2
HC CDR1
(SEQ ID NO: 3)
SYAIG
HC CDR2
(SEQ ID NO: 6)
CTSSSGDMTYYTNSVKG
HC CDR3
(SEQ ID NO: 7)
VLLEYGYVCSHPNEYDY

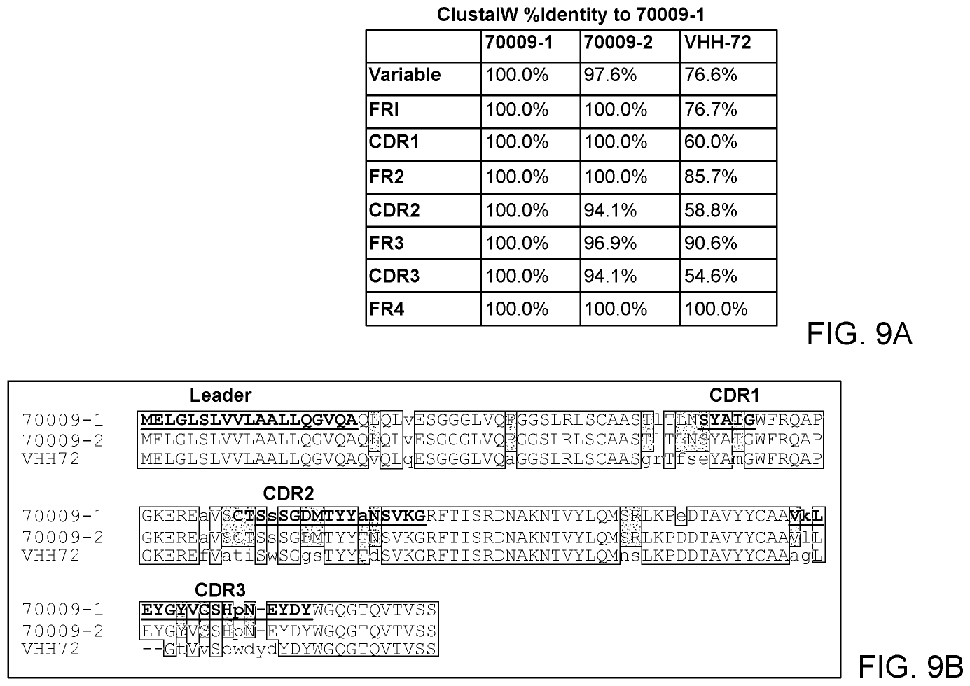

Sequence alignment of the heavy chain variable region amino acid sequences of the 70009-1, 70009-2, and VHH-72 antibodies is shown in FIG. 9B. The Kabat numbering method was used for annotation. The VHH-72 SARS-CoV-2 camelid antibody is as described in Wrapp et al. (Wrapp et al., Cell. 2020 May 28; 181(5):1004-1015.e15. doi: 10.1016/j.cell.2020.04.031). FIG. 9A presents a Clustal Identity Matrix for 70009-1, 70009-2, and VHH-72, showing ClustalW % Identity to 70009-1.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

### SEQUENCE FREE LISTING

SEQ ID NO: 1
amino acid sequence of VHH region of 70009-1 clone
SEQ ID NO: 2
Amino acid sequence of VHH region of 70009-2 clone
SEQ ID NO: 3
Amino acid sequence of the CDR1 region of the VHH region of 70009-1 and 70009-2 clones
SEQ ID NO: 4
Amino acid sequence of the CDR2 region of the 70009-1 clone
SEQ ID NO: 5
Amino acid sequence of the CDR3 region of the 70009-1 clone
SEQ ID NO: 6
Amino acid sequence of the CDR2 region of the 70009-2 clone
SEQ ID NO: 7
Amino acid sequence of the CDR3 region of the 70009-2 clone

1. An antigen binding molecule comprising an antibody variable domain with an amino acid sequence comprising:
at least 75%, at least 80%, at least 85%, at least 90%, at least 95%, at least 96%, at least 97%, at least 98%, or at least 99% sequence identity to QLQLVESGGGLVQPGGSLRLSCAASTLTLNSYAIGWFRQAPGKEREAVSCTSSSGDMTY YANSVKGRFTISRDNAKNTVYLQMSRLKPEDTAVYYCAAVKLEYGYVCSSHPNEYDYW GQGTQVTVSS (SEQ ID NO: 1); or
at least 75%, at least 80%, at least 85%, at least 90%, at least 95%, at least 96%, at least 97%, at least 98%, or at least 99% sequence identity to QLQLVESGGGLVQPGGSLRLSCAASTLTLNSYAIGWFRQAPGKEREAVSCTSSSGDMTY YTNSVKGRFTISRDNAKNTVYLQMSRLKPDDTAVYYCAAVLLEYGYVCSSHPNEYDYW GQGTQVTVSS (SEQ ID NO: 2); and
wherein the antibody binding molecule specifically binds the SARS-CoV-2 spike protein S1.

2. The antigen binding molecule of claim 1, wherein the variable domain comprises an amino acid sequence comprising
(SEQ ID NO: 1)
QLQLVESGGGLVQPGGSLRLSCAASTLTLNSYAIGWFRQAPGKEREAVS
CTSSSGDMTYYANSVKGRFTISRDNAKNTVYLQMSRLKPEDTAVYYCAA
VKLEYGYVCSHPNEYDYWGQGTQVTVSS
or
(SEQ ID NO: 2)
QLQLVESGGGLVQPGGSLRLSCAASTLTLNSYAIGWFRQAPGKEREAVS
CTSSSGDMTYYTNSVKGRFTISRDNAKNTVYLQMSRLKPDDTAVYYCAA
VLLEYGYVCSHPNEYDYWGQGTQVTVSS.

3. (canceled)

4. The antigen binding molecule of claim 1, wherein the variable domain comprises an antibody variable domain comprising one or more complementary determining regions (CDRs) selected from:
a CDR1 sequence comprising an amino acid sequence which is identical to, or differs by one, two, or three amino acids from, SYAIG (SEQ ID NO: 3);
a CDR2 sequence comprising an amino acid sequence which is identical to, or differs by one, two, three, four, or five amino acids from, CTSSSGDMTYYANSVKG (SEQ ID NO: 4) or which is identical to, or differs by one, two, three, four, or five amino acids from CTSSSGDMTYYTNSVKG (SEQ ID NO: 6); and
a CDR3 sequence comprising an amino acid sequence which is identical to, or differs by one, two, three, four, or five amino acids from, VKLEYGYVCSHPNEYDY (SEQ ID NO: 5) or which is identical to, or differs by one, two, three, four, or five amino acids from
(SEQ ID NO: 7)
VLLEYGYVCSHPNEYDY.

5. The antigen binding molecule of claim 4, wherein the antigen binding molecule comprises an antibody variable domain comprising:
a CDR1 sequence comprising the amino acid sequence SYAIG (SEQ ID NO: 3); and
a CDR2 sequence comprising the amino acid sequence CTSSSGDMTYYANSVKG (SEQ ID NO: 4) or CTSSSGDMTYYTNSVKG (SEQ ID NO: 6); and
a CDR3 sequence comprising the amino acid sequence VKLEYGYVCSHPNEYDY (SEQ ID NO: 5) or VLLEYGYVCSHPNEYDY (SEQ ID NO: 7).

6. The antigen binding molecule of claim 4, wherein the antigen binding molecule comprises an antibody variable domain comprising:
a CDR1 sequence comprising the amino acid sequence SYAIG (SEQ ID NO: 3); and
a CDR2 sequence comprising the amino acid sequence CTSSSGDMTYYANSVKG (SEQ ID NO: 4); and
a CDR3 sequence comprising the amino acid sequence VKLEYGYVCSHPNEYDY (SEQ ID NO: 5);
or comprising:
a CDR1 sequence comprising the amino acid sequence SYAIG (SEQ ID NO: 3); and
a CDR2 sequence comprising the amino acid sequence CTSSSGDMTYYTNSVKG (SEQ ID NO: 6); and
a CDR3 sequence comprising the amino acid sequence VLLEYGYVCSHPNEYDY (SEQ ID NO: 7).

7. An antibody variable domain comprising an amino acid sequence with at least 75%, at least 80%, at least 85%, at least 90%, at least 95%, at least 96%, at least 97%, at least 98%, or at least 99% sequence identity to QLQLVESGGGLVQPGGSLRLSCAASTLTLNSYAIGWFRQAPGKEREAVSCTSSSGDMTY YANSVKGRFTISRDNAKNTVYLQMSRLKPEDTAVYYCAAVKLEYGYVCSSHPNEYDYW GQGTQVTVSS (SEQ ID NO: 1) or QLQLVESGGGLVQPGGSLRLSCAASTLTLNSYAIGWFRQAPGKEREAVSCTSSSGDMTY YTNSVKGRFTISRDNAKNTVYLQMSRLKPDDTAVYYCAAVLLEYGYVCSSHPNEYDYW GQGTQVTVSS (SEQ ID NO: 2); and
wherein the antibody variable domain specifically binds the SARS-CoV-2 spike protein S1.

8-12. (canceled)

13. A camelid antibody single variable domain comprising an amino acid sequence with at least 75%, at least 80%, at least 85%, at least 90%, at least 95%, at least 96%, at least 97%, at least 98%, or at least 99% sequence identity to QLQLVESGGGLVQPGGSLRLSCAASTLTLNSYAIGWFRQAPGKEREAVSCTSSSGDMTY YANSVKGRFTISRDNAKNTVYLQMSRLKPEDTAVYYCAAVKLEYGYVCSHIPNEYDYW GQGTQVTVSS (SEQ ID NO: 1) or QLQLVESGGGLVQPGGSLRLSCAASTLTLNSYAIGWFRQAPGKEREAVSCTSSSGDMTY YTNSVKGRFTISRDNAKNTVYLQMSRLKPDDTAVYYCAAVLLEYGYVCSHIPNEYDYW GQGTQVTVSS (SEQ ID NO: 2) wherein the camelid antibody single variable domain specifically binds the SARS-CoV-2 spike protein S1.

14. The camelid antibody single variable domain of claim 13, wherein the camelid antibody single variable domain comprises an amino acid sequence comprising
(SEQ ID NO: 1)
QLQLVESGGGLVQPGGSLRLSCAASTLTLNSYAIGWFRQAPGKEREAVS
CTSSSGDMTYYANSVKGRFTISRDNAKNTVYLQMSRLKPEDTAVYYCAA
VKLEYGYVCSHPNEYDYWGQGTQVTVSS
or
(SEQ ID NO: 2)
QLQLVESGGGLVQPGGSLRLSCAASTLTLNSYAIGWFRQAPGKEREAVS
CTSSSGDMTYYTNSVKGRFTISRDNAKNTVYLQMSRLKPDDTAVYYCAA
VLLEYGYVCSHPNEYDYWGQGTQVTVSS.

15. (canceled)

16. The camelid antibody single variable domain of claim 13, wherein the camelid antibody single variable domain comprises an amino acid sequence comprises one or more complementary determining regions (CDRs) selected from:
a CDR1 sequence comprising an amino acid sequence which is identical to, or differs by one, two, or three amino acids from, SYAIG (SEQ ID NO: 3);
a CDR2 sequence comprising an amino acid sequence which is identical to, or differs by one, two, three, four, or five amino acids from, CTSSSGDMTYYANSVKG (SEQ ID NO: 4) or which is identical to, or differs by one, two, three, four, or five amino acids from CTSSSGDMTYYTNSVKG (SEQ ID NO: 6); and
a CDR3 sequence comprising an amino acid sequence which is identical to, or differs by one, two, three, four, or five amino acids from, VKLEYGYVCSHPNEYDY (SEQ ID NO: 5) or which is identical to, or differs by one, two, three, four, or five amino acids from
(SEQ ID NO: 7)
VLLEYGYVCSHPNEYDY.

17. The camelid antibody single variable domain of claim 16, comprising:
a CDR1 sequence comprising the amino acid sequence SYAIG (SEQ ID NO: 3); and
a CDR2 sequence comprising the amino acid sequence CTSSSGDMTYYANSVKG (SEQ ID NO: 4) or CTSSSGDMTYYTNSVKG (SEQ ID NO: 6); and
a CDR3 sequence comprising the amino acid sequence VKLEYGYVCSHPNEYDY (SEQ ID NO: 5) or VLLEYGYVCSHPNEYDY (SEQ ID NO: 7).

18. The camelid antibody single variable domain of claim 16 comprising:
a CDR1 sequence comprising the amino acid sequence SYAIG (SEQ ID NO: 3); and
a CDR2 sequence comprising the amino acid sequence CTSSSGDMTYYANSVKG (SEQ ID NO: 4); and
a CDR3 sequence comprising the amino acid sequence VKLEYGYVCSHPNEYDY (SEQ ID NO: 5);
or comprising:
a CDR1 sequence comprising the amino acid sequence SYAIG (SEQ ID NO: 3); and
a CDR2 sequence comprising the amino acid sequence CTSSSGDMTYYTNSVKG (SEQ ID NO: 6); and
a CDR3 sequence comprising the amino acid sequence VLLEYGYVCSHPNEYDY (SEQ ID NO: 7).

19. A camelid heavy chain IgG (hcIgG) antibody comprising a camelid antibody single variable domain of claim 13.

20. The camelid heavy chain hcIgG antibody of claim 19, comprising a llama heavy chain IgG (hcIgG) antibody.

21. (canceled)

22. The antibody binding molecule of claim 1, wherein the antibody binding molecule specifically binds to the receptor binding (RBD) domain (amino acids 319 to 541) of the SARS-CoV-2 spike protein S1.

23. The antibody binding molecule of claim 1, wherein the antibody binding molecule inhibits binding of SARS-CoV-1 and/or SARS-CoV-2 to ACE-2.

24. The antibody binding molecule of claim 23, wherein the binding of SARS-CoV-1 or SARS-CoV-2 or both SARS-CoV-1 and SARS-CoV-2 to ACE-2 is decreased by at least 10 percent (%), at least 15%, at least 20%, at least 25%, at least 30%, at least 40%, at least 50%, at least 60%, at least 70%, at least 80%, at least 90%, at least 95%, or at least 98%.

25. The antibody binding molecule of claim 1, wherein the antibody binding molecule is humanized.

26-27. (canceled)

28. The antibody binding molecule of claim 1 conjugated to a detectable marker.

29. A bispecific or multivalent antibody comprising the antibody binding molecule of claim 1.

30. A composition comprising the antibody binding molecule of claim 1.

31. The composition of claim 30, the composition further comprising at least one additional anti-SARS-CoV antibody.

32. (canceled)

33. A method comprising administering the antibody binding molecule of claim 1 to a subject.

34. The method of claim 33, wherein the subject:
is suspected of having SARS-CoV-1 or SARS-CoV-2;
has been diagnosed with SARS-CoV-1 or SARS-CoV-2; or
has been exposed to SARS-CoV-1 or SARS-CoV-2.

35-37. (canceled)

38. A method comprising using the antibody binding molecule, of claim 1 to diagnose a subject with SARS-CoV-1 or SARS-CoV-2.

39. A nucleotide sequence encoding the antibody binding molecule, of claim 1.

40-43. (canceled)
